(19) United States
(12) Patent Application Publication
(10) Pub. No.: US 2026/0179785 A1
(43) Pub. Date: Jun. 25, 2026

(54) COMPUTERIZED SYSTEMS AND METHODS FOR PERSONALIZED MEDICAL EDUCATION AND CLINICAL WORKFLOW AUTOMATION USING REAL-TIME CLINICAL DATA

(71) Applicant: Ali Borhan, Paradise Valley, AZ (US)

(72) Inventor: Ali Borhan, Paradise Valley, AZ (US)

(21) Appl. No.: 19/427,611

(22) Filed: Dec. 19, 2025

Related U.S. Application Data
(60) Provisional application No. 63/737,307, filed on Dec. 20, 2024.
(60) Provisional application No. 63/931,897, filed on Dec. 5, 2025.

(51) Int. Cl. G16H 70/00; G16H 10/60

(52) U.S. Cl. CPC G16H 70/00; G16H 10/60

(57) ABSTRACT

A computerized system for personalized medical education, compliance verification, and clinical workflow automation uses sensor-based identification and multi-modal monitoring to verify user presence, engagement, and comprehension during delivery of educational modules and required disclosures. Artificial intelligence models interpret electronic medical record data to select and sequence patient-specific content, determine procedure-specific and jurisdiction-specific compliance requirements, and adapt presentation in real time through device-level control actions including pausing, prompting, repeating, and module recalibration when noncompliance is detected. The system further supports conducting interactive clinical encounters in lieu of or in conjunction with clinician visits, including structured intake, decision support, and generation of audit-ready, billable encounter documentation under clinician oversight and safety guardrails. The system generates time-stamped interaction logs, medical decision-making and time-on-task summaries, and electronic medical record-ready outputs with provenance and compliance verification, suitable for reimbursement and audit defensibility across multiple specialties.

### CROSS-REFERENCE TO RELATED APPLICATIONS

This application claims the benefit of and priority to U.S. Provisional Application No. 63/931,897, filed Dec. 5, 2025, and also claims the benefit of and priority to U.S. Provisional Application No. 63/737,307, filed Dec. 20, 2024, which are incorporated herein by reference in its entirety.

### FIELD OF THE DISCLOSURE

The present disclosure relates to computerized systems and methods for interactive clinical encounters, personalized medical education, and automated clinical workflow and documentation using real-time clinical data.

### BACKGROUND

In busy clinical environments, clinician time is limited. Many components of a patient visit are repetitive and documentation-heavy, which reduces the time available for higher-acuity decision-making and patient counseling. Fragmented platforms for operations such as history intake, review of systems capture, standardized risk disclosures, education delivery, comprehension checks, consent-support artifact capture, and preparation of encounter documentation for electronic medical record insertion and reimbursement support, typically rely on manual clinician effort. This reliance increases burden, introduces variability, and can delay completion of required documentation and compliance tasks. Moreover, existing systems fail to automate encounter components in a billable, auditable way. For example, such systems often do not reliably produce time-stamped interaction records, defensible medical decision-making and time-on-task summaries, and electronic medical record-ready documentation with comprehensive provenance and compliance verification under appropriate clinical and legal guardrails.

Preoperative patient-facing educational modules such as training videos are increasingly used in medical settings to educate patients about upcoming surgical procedures. These videos typically cover important aspects such as the nature of the procedure, potential risks, and post-operative care instructions. Understanding and acknowledging this information is important for ensuring informed consent, reducing the risk of surgical complications, and complying with insurance-related requirements.

However, there are significant challenges in ensuring that patients actually watch and comprehend these educational modules. In busy environments such as waiting rooms, distractions can prevent patients from fully engaging with the content. Additionally, hospitals and clinics must ensure that patients have properly understood the information, which may be a requirement for legal and insurance compliance. Current systems rely heavily on manual oversight or self-reporting, both of which introduce opportunities for error and misreporting.

Currently, there is no conventional system that can automatically determine a type of educational module to present, track a patient's engagement with educational module content, verify comprehension, and complete the necessary compliance documentation in real-time, while maintaining patient confidentiality and complying with data privacy regulations. The lack of such a system creates a problem for both healthcare providers and patients, as misunderstanding about the procedure could lead to poor outcomes, legal liability, or insurance denials. This problem is further compounded by the need to access patient-specific medical records and determine which insurance-related forms must be filled out based on the individual patient's procedure.

Therefore, there is a need in the art for a computerized system that ensures patient compliance with required educational assessments while also automating clinical encounter components, documentation, and audit-ready workflows in an efficient, confidential, and secure manner.

### SUMMARY

The present disclosure describes a comprehensive computerized system that revolutionizes how healthcare providers ensure patients properly understand and engage with important medical educational content. Unlike traditional approaches that rely on manual oversight or patient self-reporting, the system described herein provides automated, real-time verification that patients are watching, comprehending, and complying with required educational modules such as preoperative videos and procedural instructions. In some embodiments, the system operates through a multi-step process that begins with user identification using at least one sensor, which may employ advanced biometric technologies including facial recognition, fingerprint scanning, voice recognition, and continuous presence verification, to ensure the correct individual is participating in the educational process.

Once a patient is identified, in some embodiments, the system associates a medical record with the user based on the identification, automatically accessing relevant clinical information to understand the patient's specific medical needs, procedure requirements, and risk factors. In some embodiments, the system then determines one or more content modules to be displayed on a client device based on the medical record, using artificial intelligence to select personalized educational content tailored to the patient's specific procedure, medical history, demographics, and literacy level. For each selected module, in some embodiments, the system determines compliance requirements, which may vary based on the type of medical procedure, jurisdiction-specific regulations, insurance requirements, and institutional policies. In some embodiments, identifying the compliance requirements includes identifying a procedure type and the jurisdiction of the procedure type, where the system executes rule-based systems in conjunction with one or more artificial intelligence models to determine regulations for specific compliance requirements associated with the jurisdiction.

In some embodiments, the system is configured to execute monitoring protocols while each content module is being displayed on a client device, implementing advanced sensor fusion technology that combines multiple monitoring methods including gaze tracking that calculates precise gaze coordinates to determine whether the user is looking at relevant screen areas. In some embodiments, attention span assessment is obtained by analyzing the duration and frequency of user gaze toward the client device, and engagement scoring based on comprehensive sensor data including facial presence detection, head orientation, blink rates, body posture, and/or ambient noise analysis. In some embodiments, the monitoring protocols include real-time presence tracking using artificial intelligence to ensure continuous user engagement. In some embodiments, identifying the user includes continuous monitoring while the one or more content modules are being displayed. In some embodiments, continuous monitoring includes executing real-time presence tracking using one or more sensors with artificial intelligence used to execute the real-time presence tracking.

In some embodiments, the system continuously determines if the compliance requirements are being met based on monitoring protocols, comparing analysis results for monitored parameters against predetermined compliance threshold values. In some embodiments, the system is configured to implement only the monitoring protocols associated with the compliance requirements, optimizing computational resources while maintaining comprehensive oversight. In some embodiments, the system is configured to flag low engagement when multiple signals suggest inattention, allowing the system to respond dynamically to patient behavior in real-time.

When noncompliance is detected, in some embodiments, the system controls the client device through various adaptive interventions, including automatically pausing content when a user's gaze moves away from the screen, presenting interactive activities responsive to drops in engagement, and implementing personalized content delivery responsive to noncompliance determinations. In some embodiments, controlling the client device includes module recalibration that adjusts one or more of content selection, sequencing, segment length, and tone in real-time to maintain patient engagement and comprehension. This dynamic response capability represents a significant advancement over conventional systems that cannot automatically adapt to individual patient needs and engagement patterns.

In some embodiments, the system incorporates several interconnected modules working in concert, including a data ingestion module that processes electronic medical records and real-time events, an adaptive decision engine functioning as a “digital twin” that maintains patient-specific decision states and orchestrates personalized content delivery. In some embodiments, an adaptive content engine that assembles multimedia, multilingual educational modules with interactive branching logic, a safety module that enforces physician-approved templates, institutional guidelines, and clinical/legal guardrails. In some embodiments, an interactive patient interface is configured to be deployable across mobile devices, tablets, desktops, and kiosks. In some embodiments, a documentation and coding engine that produces EMR-ready documentation with comprehensive audit trails.

In some embodiments, the system supports comprehensive multi-specialty deployment across urology, cardiology, gastroenterology, oncology, obstetrics/gynecology, and orthopedics, among others, where each specialty module utilizes the same core pipeline while incorporating specialty-specific content libraries, risk models, and clinical pathways.

In some embodiments, upon completion of educational modules, the system automatically updates portions of a medical database by compiling monitoring results and inserting the monitoring results into the medical record, creating comprehensive audit trails that document patient education delivery, comprehension evidence, consent support, and compliance verification.

In some embodiments, the system employs multiple artificial intelligence models including natural language processing for medical record interpretation, computer vision for gaze and pose detection, predictive analytics for risk assessment, and reinforcement learning for optimal content sequencing, all working under strict safety guardrails to ensure clinical accuracy and patient safety.

In some embodiments, the system conducts a clinical encounter in lieu of a traditional clinician visit. For example, the system may initiate and guide an encounter, collect history and symptom inputs, present risk-adjusted disclosures and instructions, enforce clinical and legal guardrails, and compile encounter-ready documentation for insertion into an electronic medical record, with provider review, acknowledgment, or sign-off occurring asynchronously or synchronously.

In some embodiments, the system substitutes for portions of clinician time while maintaining clinician oversight and safety guardrails. Non-limiting examples include substituting time associated with patient education, history intake, risk disclosure, comprehension verification, consent artifact capture, and preparation of encounter documentation, with provider review, approval workflows, and escalation controls preserved.

In some embodiments, the extent of automated clinical interaction provided by the system is configurable and may encompass partial, majority, or near-complete automation of encounter flow, subject to clinical and legal guardrails and without autonomous diagnosis or treatment determinations. Automation may range from education and knowledge checkpoints to structured intake (e.g., history, symptoms, review of systems), triage and escalation proposals, and generation of encounter documentation, with provider override preserved. The extent of automation may evolve over time as technology, data sources, and applicable approvals advance, under audited, versioned update processes.

In some embodiments, when an encounter is conducted in lieu of a traditional visit, the system produces encounter records aligned to payer documentation requirements for reimbursable services rendered via the system, including, without limitation, problem(s) addressed, data reviewed, risk assessment elements, and time-on-task summaries, and outputs electronic medical record-ready documentation for insertion into the patient record.

In some embodiments, the system supports digital evaluation/management encounters and care management services by generating encounter-ready documentation that reflects patient-specific interactions, problem, data, and risk elements, and time-based activity, and by incorporating remote and wearable signals and longitudinal guidance. In some embodiments, these capabilities operate with multilingual and accessibility adaptations and are governed by safety guardrails prior to delivery and electronic medical record insertion.

In some embodiments, disclosed subject matter may encompass compliance-verified education systems and methods, interactive clinical encounter and visit-substitution systems and methods, and automated documentation and coding systems and methods, each anchored to sensor-based identification and monitoring, client-device control actions, and electronic medical record write-back. In some embodiments, one or more non-transitory computer-readable media store instructions which, when executed by one or more processors, implement any of the foregoing aspects under clinical and legal guardrails, as further described below.

### DESCRIPTIONS OF THE DRAWINGS

The features, and advantages of the disclosure will be apparent from the following description of embodiments as illustrated in the accompanying drawings, in which reference characters refer to the same parts throughout the various views. The drawings are not necessarily to scale, emphasis instead being placed upon illustrating principles of the disclosure:

FIG. 1 shows a block diagram of a suitable non-limiting example framework within which the systems and methods disclosed herein are implemented, according to some embodiments of the present disclosure;

FIG. 2 shows a block diagram illustrating further components of the framework, according to some embodiments of the present disclosure;

FIG. 3 illustrates a non-limiting system workflow, according to some embodiments of the present disclosure;

FIG. 4 illustrates a non-limiting AI execution workflow, according to some embodiments of the present disclosure;

FIG. 5 depicts a non-limiting example implementation of the framework according to some embodiments of the present disclosure;

FIG. 6 depicts a non-limiting implementation of the framework, according to some embodiments of the present disclosure;

FIG. 7 shows a block diagram illustrating a computing device showing an non-limiting example of a client or server device, according to some embodiments of the present disclosure;

FIG. 8 illustrates a non-limiting system architecture for a compliance engine, according to some embodiments of the present disclosure.

FIG. 9 depicts a data ingestion and normalization pipeline, according to some embodiments of the present disclosure.

FIG. 10 shows internal components of an adaptive decision engine, according to some embodiments of the present disclosure.

FIG. 11 illustrates internal components of the adaptive content engine, according to some embodiments of the present disclosure.

FIG. 12 depicts a real-time personalization loop, according to some embodiments of the present disclosure.

FIG. 13 shows provider-facing views within an interactive patient interface, according to some embodiments of the present disclosure.

FIG. 14 illustrates internal components of a documentation and coding engine, according to some embodiments of the present disclosure.

FIG. 15 depicts multi-specialty deployment of a compliance engine, according to some embodiments of the present disclosure;

FIG. 16 shows a cataloged index of educational modules within the adaptive content engine, according to some embodiments of the present disclosure.

FIG. 17 illustrates knowledge-checkpoint panels within the interactive patient interface, according to some embodiments of the present disclosure.

FIG. 18 depicts a signature capture panel within the interactive patient interface, according to some embodiments of the present disclosure.

### DETAILED DESCRIPTION

According to some embodiments, the system described herein provides integrated control and management of patient education for any type of medical information that requires pre-education, concurrent education, and/or post-education delivery. In some embodiments, the system is configured to adapt educational content for routine office visits, specialty consultations, diagnostic procedures, therapeutic interventions, surgical procedures, chronic disease management, preventive care screenings, medication counseling, lifestyle modification guidance, and ongoing post-treatment follow-up, as non-limiting examples. In some embodiments, the educational materials may include, without limitation, animated tutorials, procedural simulations, risk-benefit explanations, medication instructions, symptom monitoring guidance, recovery timelines, preventive care recommendations, or any combination thereof.

In some embodiments, the system is configured to receive and process any type of clinical information available from electronic medical record systems, including, but not limited to, laboratory results, imaging findings, pathology reports, medication lists, vital signs, clinical notes, appointment data, and demographic information. According to some embodiments, this clinical data is used to dynamically customize system presented educational modules, adjust content depth and complexity, modify risk explanations, personalize recovery instructions, and sequence educational pathways based on individual patient characteristics and real-time clinical status.

The selection and presentation of specific educational content is determined by intelligent analysis of EMR-derived information, according to some embodiments. For example, in some embodiments, diagnostic codes may automatically trigger condition-specific educational modules, such as diabetes management pathways for newly diagnosed diabetic patients, hypertension education for cardiovascular conditions, or procedural preparation modules for scheduled interventions. In some embodiments, laboratory values influence content selection and emphasis, where elevated cardiac enzymes may trigger heart disease education modules, abnormal kidney function may modify medication counseling content, or concerning tumor markers may initiate oncology-specific educational pathways. According to some embodiments, medication lists drive personalized pharmaceutical education, including drug-specific side effect monitoring, interaction warnings, and adherence strategies tailored to the patient's complete medication profile.

In some embodiments, imaging results and pathology findings automatically modify educational content to reflect current clinical status, such as adapting surgical education modules based on imaging-determined procedural complexity, or adjusting cancer education pathways following pathological staging results. According to some embodiments, patient demographic information including age, language preferences, literacy levels, and cultural background influences content presentation style, complexity, and delivery modality. In some embodiments, the system is configured to consider temporal factors such as appointment scheduling, procedure timing, and recovery phases to sequence educational content appropriately, delivering pre-visit preparation materials in advance of appointments, procedural education prior to scheduled interventions, and recovery-specific guidance during appropriate post-treatment phases.

In some embodiments, comorbidity profiles derived from problem lists and medication histories influence educational content emphasis and risk stratification. For example, patients with diabetes receiving surgical education may receive enhanced wound healing information, while patients with cardiac conditions undergoing procedures may receive specialized perioperative cardiac risk education. According to some embodiments, the system continuously analyzes combinations of EMR data elements to determine optimal educational pathways, such as combining diagnostic codes, laboratory trends, medication changes, and appointment scheduling to create personalized, temporally-appropriate educational experiences.

In some embodiments, the disclosed system includes monitoring and feedback execution configured to track patient engagement, comprehension, and interaction patterns during educational module delivery. In some embodiments, these monitoring capabilities enable real-time adaptation of content presentation, automatic adjustment of educational sequences, and continuous refinement of personalization algorithms based on observed patient behavior and clinical outcomes. According to some embodiments, the system creates closed-loop feedback cycles where patient responses and EMR updates continuously inform and improve subsequent educational content delivery, ensuring that educational interventions remain clinically relevant and personally meaningful throughout the entire spectrum of medical care.

In some embodiments, the comprehensive educational and clinical workflow capabilities described above are achieved through an interconnected system architecture comprising multiple engines, modules, and processing layers, as illustrated in the accompanying figures. In a non-limiting framework, according to some embodiments, the system includes a data ingestion layer that normalizes and processes EMR information, an adaptive decision engine that maintains a continuously updated computational representation of each patient, an adaptive content generation engine that assembles personalized multimedia educational modules, a hybrid safety layer that ensures clinical accuracy and compliance, an interactive patient interface that delivers content and captures patient responses, and a documentation and coding engine that automates clinical record generation. In some embodiments, these components operate within a real-time personalization loop that continuously updates patient models and educational content based on new EMR data and patient interactions. According to some embodiments, the modular architecture enables deployment across multiple medical specialties while maintaining unified technical infrastructure and standardized clinical workflows. The following detailed description demonstrates how the broad spectrum of medical education and clinical automation capabilities are technically implemented and deployed in clinical practice, in accordance with some embodiments.

According to some embodiments, the disclosed system is configured to perform interactive clinical encounter functions (e.g., clinical workflow automation), extending beyond patient education to encompass comprehensive clinical interactions. In some embodiments, the system may operate in lieu of a clinician visit, in conjunction with a clinician visit, or adjacent to a clinician visit, providing flexible deployment modalities that adapt to various healthcare delivery contexts. According to some embodiments, the system may substitute for portions of clinician time while maintaining clinician oversight and safety guardrails (e.g., via a safety module), where the substitution includes structured clinical questioning, symptom assessment, and decision support activities traditionally performed during face-to-face encounters. In some embodiments, the interactive clinical encounter functions include automated history-taking, review of systems documentation, and preliminary clinical assessments that are subsequently reviewed and validated by clinicians through provider-facing views within an interactive patient interface.

In some embodiments, the extent of automated clinical interaction may increase over time as technology capabilities, data sources, and regulatory approvals evolve, where the system is configured to adapt its automation scope without requiring fundamental architectural changes. According to some embodiments, the system preserves coverage for partial visit automation, majority visit automation, or near-complete visit automation without asserting autonomous diagnosis or treatment, maintaining appropriate clinical oversight boundaries. In some embodiments, an adaptive decision engine is configured to expand its clinical reasoning capabilities as new artificial intelligence models become available and regulatory frameworks permit enhanced automation, while the safety module enforces evolving clinical and legal guardrails that correspond to the current regulatory environment.

In some embodiments, documentation outputs produced by a documentation and coding engine are sufficient to support billing for services rendered via the system, where the documentation includes comprehensive encounter records that satisfy payer requirements and regulatory standards. In some embodiments, the system supports billing in lieu of a traditional visit and in conjunction with a clinician visit, providing flexible reimbursement pathways that align with various healthcare delivery models. In some embodiments, billing support is tied to time-based services, medical decision-making (MDM) elements, digital evaluation and management encounters, remote monitoring activities, and care management functions, where time-based coding calculator module aggregates interaction durations and MDM extractor module identifies billable clinical decision elements. In some embodiments, the system generates encounter documentation that supports current procedural terminology coding frameworks and evaluation and management service classifications without explicitly referencing specific billing codes, maintaining flexibility for evolving reimbursement methodologies.

In some embodiments, the system avoids over-reliance on any single sensor modality such as gaze tracking, instead implementing multi-modal interaction and sensor fusion that combines structured questioning, user inputs, and data ingestion from multiple sources. According to some embodiments, sensor fusion combines multiple modalities including facial recognition, voice recognition, motion detection, proximity sensing, and interactive response capture to compute comprehensive engagement metrics and clinical interaction assessments. In some embodiments, the system incorporates structured clinical questioning through the interactive patient interface, symptom intake workflows, and comprehensive data ingestion via data ingestion module, ensuring that clinical assessments do not depend solely on passive monitoring but include active patient participation and structured clinical interactions.

According to some embodiments, the system generates audit-ready, time-stamped records of clinical interactions, patient comprehension verification, and consent processes through a documentation and coding engine, where the audit trails include comprehensive provenance tracking from data ingestion through clinical decision-making and documentation output. In some embodiments, audit trails are tied to legal defensibility, regulatory compliance, and reimbursement defensibility, providing comprehensive documentation that supports clinical liability protection, regulatory audit requirements, and payer validation processes. According to some embodiments, an interaction log module captures complete timestamped records that include clinical reasoning elements, patient response validation, and consent support documentation that collectively establish legal and regulatory compliance for clinical encounters.

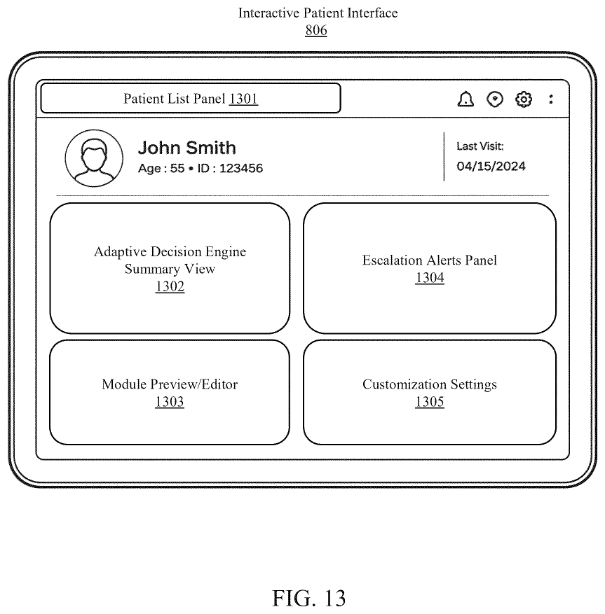

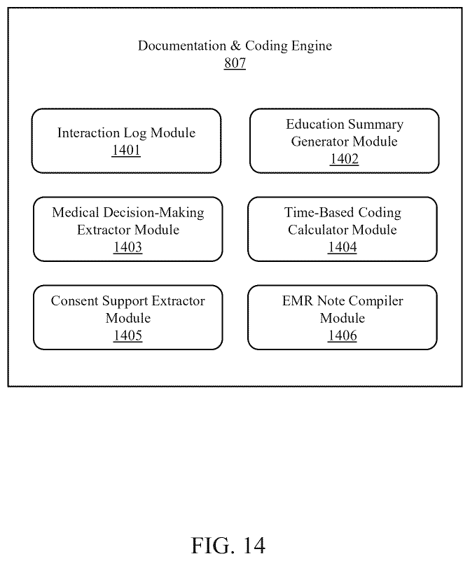

In some embodiments, the system reinforces the end-to-end architecture supporting clinical encounter documentation and auditability, where a compliance engine provides comprehensive clinical workflow capabilities extending beyond educational compliance to encompass full encounter management and documentation generation. In some embodiments, a real-time personalization loop implements device-level control that provides statutory subject matter anchoring, where the loop demonstrates concrete device control actions and technical improvements to clinical workflow automation. According to some embodiments, FIG. 13 clarifies interactive clinical questioning and symptom intake capabilities within the provider-facing views of interactive patient interface, extending beyond educational content delivery to encompass structured clinical assessment workflows. In some embodiments, FIG. 14 emphasizes visit-level documentation generation and billable encounter output through documentation and coding engine 807, where the system produces comprehensive clinical encounter records that support various reimbursement and regulatory requirements for digitally-delivered healthcare services.

According to some embodiments, the system implements an end-to-end architecture that supports comprehensive clinical encounter documentation and auditability, where a compliance engine provides clinical workflow capabilities extending beyond educational compliance to encompass full encounter management and documentation generation. In some embodiments, the system executes a real-time personalization loop as device-level control functionality that provides concrete device control actions and technical improvements to clinical workflow automation, where the loop enables direct client device manipulation and EMR integration. In some embodiments, the system performs interactive clinical questioning and symptom intake capabilities through provider-facing views within the interactive patient interface, extending beyond educational content delivery to encompass structured clinical assessment workflows that support comprehensive patient evaluation. In some embodiments, the system generates visit-level documentation and billable encounter output through a documentation and coding engine, where the system produces comprehensive clinical encounter records that support various reimbursement and regulatory requirements for digitally-delivered healthcare services.

In some embodiments, the system architecture supports interactive clinical encounters and visit substitution capabilities where the system performs a majority or entirety of clinical visits through structured history of present illness documentation, review of captured items, symptom triage, and decision support with provider review and escalation workflows. According to some embodiments, the system encompasses documentation, coding, and billing automation through automated medical decision-making extraction, time-based documentation generation, payer-aware and jurisdiction-aware encounter records, and audit-ready billing support that enables revenue generation and enterprise licensing opportunities. In some embodiments, the system supports adaptive digital twin functionality and longitudinal care management through persistent patient-specific decision states maintained by adaptive decision engine, cross-visit learning and personalization capabilities, and longitudinal risk prediction and guidance systems that enable chronic care management and follow-up monetization. In some embodiments, the system enables avatar-driven and multimodal clinical interface capabilities through avatar-guided clinical encounters, voice-based and multimodal interaction modalities, and accessibility-enhanced multilingual visit delivery systems that support global deployment and clinical differentiation strategies.

The present disclosure will now be described more fully hereinafter with reference to the accompanying drawings, which form a part hereof, and which show, through non-limiting illustrations, certain example embodiments. Subject matter may, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware, or any combination thereof (other than software per se). The following detailed description is, therefore, not intended to be taken in a limiting sense.

Throughout the specification and claims, terms may have nuanced meanings suggested or implied in context beyond an explicitly stated meaning. Likewise, the phrase “in some embodiments” as used herein does not necessarily refer to the same embodiment and the phrase “in some embodiments” as used herein does not necessarily refer to a different embodiment. It is intended, for example, that claimed subject matter include combinations of various features of the single system described herein, in whole or in part, in accordance with some embodiments.

In general, terminology may be understood at least in part from usage in context. For example, terms, such as “and,” “or,” or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures, or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

“Substantially” and “approximately” when used in conjunction with a value encompass a difference of 5% or less of the same unit and/or scale of that being measured.

The terms “simultaneously” and “real-time” as used herein include lag and/or latency times associated with a conventional and/or proprietary computer, such as processors and/or networks described herein attempting to process multiple types of data at the same time. “Simultaneously” and “real-time” also include the time it takes for digital signals to transfer from one physical location to another, be it over a wireless and/or wired network, and/or within processor circuitry.

As used herein, “can” or “may” or derivations there of (e.g., the system display can show X) are used for descriptive purposes only and is understood to be synonymous and/or interchangeable with “configured to” (e.g., the computer is configured to execute instructions X) when defining the metes and bounds of the system. The phrase “configured to” also denotes the step of configuring a structure or computer to execute a function according to some embodiments.

The present disclosure is described below with reference to block diagrams and operational illustrations of methods and devices. It is understood that each block of the block diagrams or operational illustrations, and combinations of blocks in the block diagrams or operational illustrations, can be implemented by means of analog or digital hardware and computer program instructions. These computer program instructions can be provided to a processor of a computer to alter its function as detailed herein. Instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

For the purposes of this disclosure a non-transitory computer readable medium (or computer-readable storage medium/media) stores computer data, which data can include computer program code (or computer-executable instructions) that is executable by a computer, in machine readable form. By way of example, and not limitation, a computer readable medium may include computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and non-removable media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, optical storage, cloud storage, magnetic storage devices, or any other physical or material medium which can be used to tangibly store the desired information or data or instructions and which can be accessed by a computer or processor.

For the purposes of this disclosure the term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

For the purposes of this disclosure a “network” should be understood to refer to a network that may couple devices so that communications may be exchanged, such as between a server and a client device or other types of devices, including between wireless devices coupled via a wireless network, for example. A network may also include mass storage, such as network attached storage (NAS), a storage area network (SAN), a content delivery network (CDN) or other forms of computer or machine-readable media, for example. A network may include the Internet, one or more local area networks (LANs), one or more wide area networks (WANs), wire-line type connections, wireless type connections, cellular or any combination thereof. Likewise, sub- networks, which may employ differing architectures or may be compliant or compatible with differing protocols, may interoperate within a larger network.

For purposes of this disclosure, a “wireless network” should be understood to couple client devices with a network. A wireless network may employ stand-alone ad-hoc networks, mesh networks, Wireless LAN (WLAN) networks, cellular networks, or the like. A wireless network may further employ a plurality of network access technologies, including Wi-Fi, Long Term Evolution (LTE), WLAN, Wireless Router mesh, or 2nd, 3rd, 4^{th} or 5^{th} generation (2G, 3G, 4G or 5G) cellular technology, mobile edge computing (MEC), Bluetooth®, 802.11b/g/n, or the like. Network access technologies may enable wide area coverage for devices, such as client devices with varying degrees of mobility, for example.

In short, a wireless network may include virtually any type of wireless communication by which signals may be communicated between devices, such as a client device or a computing device, between or within a network, or the like.

A computing device, which may include one or more computers, may be capable of sending or receiving signals, such as via a wired or wireless network, or may be capable of processing or storing signals, such as in memory as physical memory states, and may, therefore, operate as a server. Thus, devices capable of operating as a server may include, as examples, dedicated rack-mounted servers, desktop computers, laptop computers, set top boxes, integrated devices combining various features, such as two or more features of the foregoing devices, or the like.

For purposes of this disclosure, a client (or user, entity, subscriber or customer) device may include a computing device capable of sending or receiving signals, such as via a wired or a wireless network. A client device may, for example, include a desktop computer or a portable device, such as a cellular telephone, a smart phone, a display pager, a radio frequency (RF) device, an infrared (IR) device a Near Field Communication (NFC) device, a Personal Digital Assistant (PDA), a handheld computer, a tablet computer, a phablet, a laptop computer, a television, a wearable computer, smart watch, and/or an integrated or distributed device combining various features, such as features of the forgoing devices, or the like.

A client device may vary in terms of capabilities or features. Claimed subject matter is intended to cover a wide range of potential variations, such as a web-enabled client device or previously mentioned devices may include a high-resolution screen (HD or 4K for example), one or more physical or virtual keyboards, mass storage, one or more accelerometers, one or more gyroscopes, global positioning system (GPS) or other location-identifying type capability, or a display with a high degree of functionality, such as a touch-sensitive color 2D or 3D display, for example.

Certain embodiments and principles will be discussed in more detail with reference to the figures. According to some embodiments, the disclosed framework provides integrated control and management of one or more devices and/or the applications executing thereon.

According to some embodiments, the discussion herein may focus on embodiments related to electronic monitoring of user interactions with client devices for medical purposes; however, these examples should not be construed as limiting, as one of skill in the art would understand that the disclosed framework described herein can apply to various scenarios without departing from the scope of the instant disclosure. For example, in a professional environment, the system may be configured to monitor compliance in testing environments. In some embodiments, the system may be applied to vehicles or aircraft, to monitor, evaluate, record, and control equipment based on monitored compliance, as non-limiting examples.

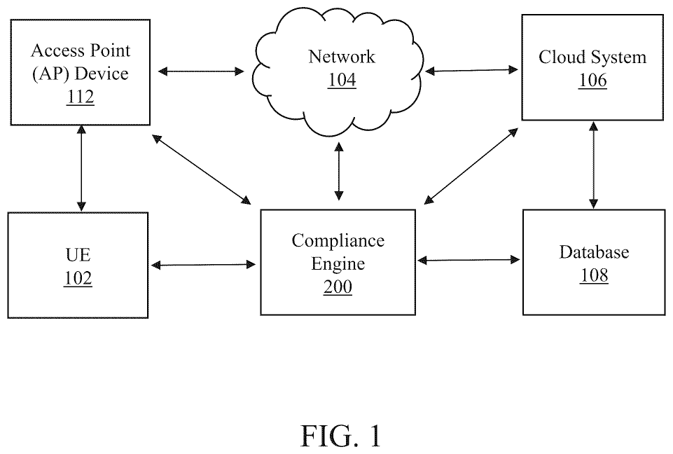

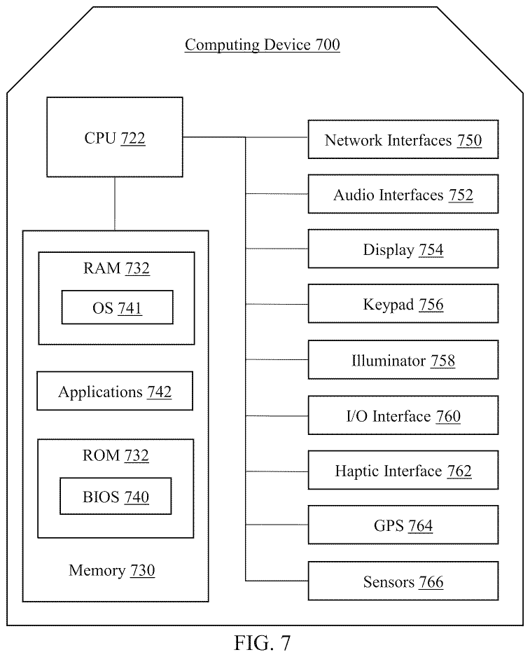

With reference to FIG. 1, system 100 is depicted which includes user equipment (UE) 102 (e.g., a client device, as mentioned above and discussed below in relation to FIG. 7), access point (AP) device 112, network 104, cloud system 106, database 108 and compliance engine 200. It should be understood that while system 100 is depicted as including such components, it should not be construed as limiting, as one of ordinary skill in the art would readily understand that varying numbers of UEs, AP devices, peripheral devices, cloud systems, databases and networks can be utilized. However, for purposes of explanation, system 100 is discussed in relation to the example depiction in FIG. 1.

According to some embodiments, UE 102 can be any type of device, such as, but not limited to, a mobile (smart) phone, tablet, laptop, sensor, IoT device, autonomous machine, appliance, and/or any other device equipped with a cellular and/or wireless or wired transceiver. For example, UE 102 can be a smart phone with program applications (Apps) installed that can execute the compliance monitoring system, which as discussed below in more detail, can enable the identification and/or collection of compliance activity information.

In some embodiments, one or more peripheral devices (not shown) can be connected to UE 102, and can be any type of peripheral device, such as, but not limited to, a wearable device (e.g., smart watch), printer, speaker, sensor, and the like. In some embodiments, peripheral device can be any type of device that is connectable to UE 102 via any type of known or to be known pairing mechanism, including, but not limited to, Wi-Fi, Bluetooth™, Bluetooth Low Energy (BLE), NFC, and the like. For example, the peripheral device can be a second client device (e.g., computer monitor) that connectively pairs with UE 102, which is a user's smart phone in some non-limiting examples, so that additional audio and visual monitoring can be observed simultaneously with execution of the system monitoring functions in accordance with some embodiments.

According to some embodiments, AP device 112 is a device that creates a wireless local area network (WLAN) for the location. According to some embodiments, the AP device 112 can be, but is not limited to, a router, switch, hub and/or any other type of network hardware that can project a Wi-Fi signal to a designated area. In some embodiments, UE 102 may include an AP device.

In some embodiments, network 104 can be any type of network, such as, but not limited to, a wireless network, cellular network, the Internet, and the like (as discussed above). Network 104 facilitates connectivity of the components of system 100, as illustrated in FIG. 1. In some embodiments, the system applies encryption in transit and at rest, role-based access controls, comprehensive audit logging of protected health information access and safety actions, and field-level provenance for data elements and documentation, aligned with safety module 805 guardrails and documentation and coding engine 807 practices.

According to some embodiments, cloud system 106 may be any type of cloud operating platform and/or network-based system upon which applications, operations, and/or other forms of network resources may be located. For example, system 106 may be a service provider and/or network provider from where services and/or applications may be accessed, sourced, or executed from. For example, system 106 can represent the cloud-based architecture associated with a smart home or network provider, which has associated network resources hosted on the internet or private network (e.g., network 104), which enables (via engine 200) the device control and management discussed herein.

In some embodiments, cloud system 106 may include a server(s) and/or a database of information which is accessible over network 104. In some embodiments, a database 108 of cloud system 106 may store a dataset of data and metadata associated with local and/or network information related to a user(s) of the components of system 100 and/or each of the components of system 100 (e.g., UE 102, AP device 112, and the services and applications provided by cloud system 106 and/or compliance engine 200).

In some embodiments, for example, cloud system 106 can provide a private/proprietary management platform, whereby engine 200, discussed infra, corresponds to the novel functionality system 106 enables, hosts and provides to a network 104 and other devices/platforms operating thereon.

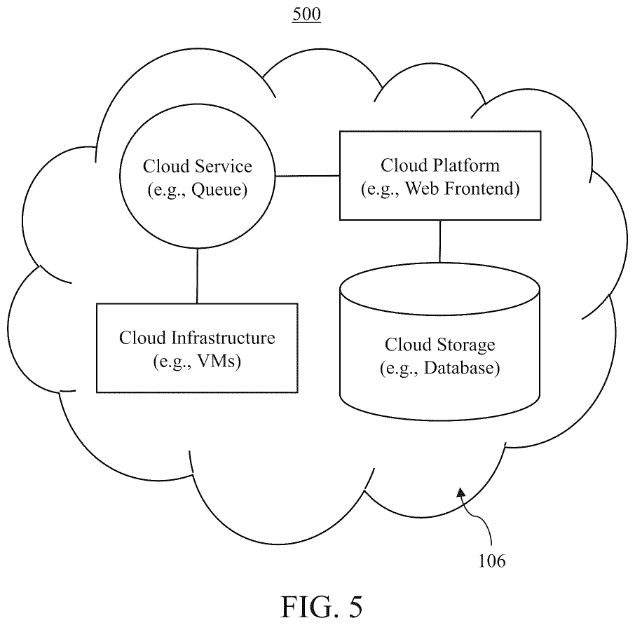

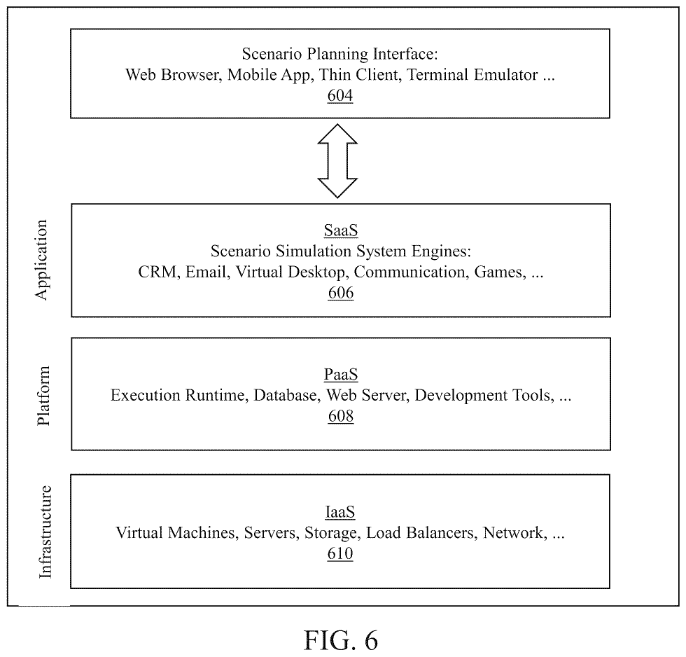

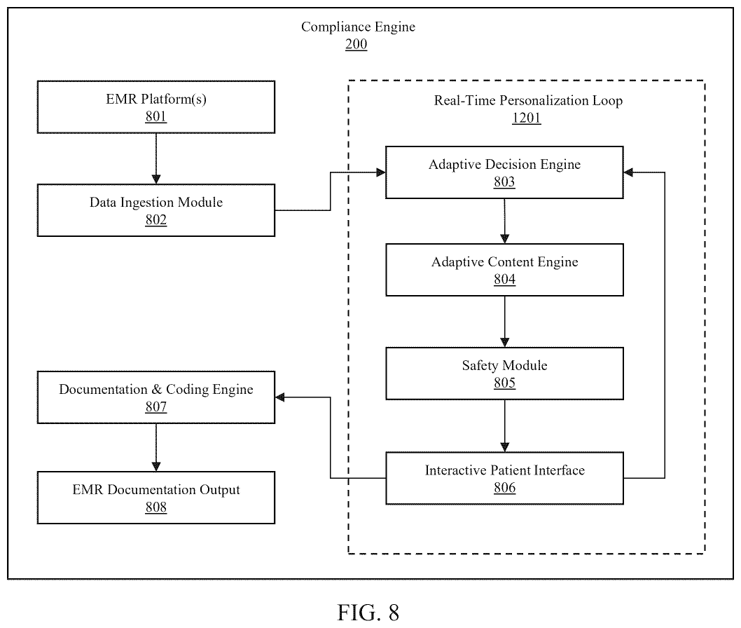

Turning to FIGS. 5 and 6, in some embodiments, the exemplary computer-based systems/platforms, the exemplary computer-based devices, and/or the exemplary computer-based components of the present disclosure may be specifically configured to operate in a cloud computing/architecture 120 such as, but not limiting to: infrastructure as a service (IaaS) 610, platform as a service (PaaS) 608, and/or software as a service (SaaS) 606 using a web browser, mobile app, thin client, terminal emulator or other endpoint 604. FIGS. 5 and 6 illustrate schematics of non-limiting implementations of the cloud computing/architecture(s) in which the exemplary computer-based systems for administrative customizations and control of network-hosted application program interfaces (APIs) of the present disclosure may be specifically configured to operate. As further detailed in FIGS. 8-11, the cloud platform and services may host data ingestion module 802, adaptive decision engine 803, adaptive content engine 804, safety module 805, interactive patient interface 806, and documentation and coding engine 807 within the IaaS/PaaS/SaaS deployment. As further detailed in FIGS. 8-11 and 13, the application endpoints correspond to interactive patient interface 806, while SaaS/PaaS/IaaS tiers host adaptive content engine 804, adaptive decision engine 803, and data ingestion module 802 under governance by safety module 805 and with documentation by engine 807.

Turning back to FIG. 1, according to some embodiments, database 108 may correspond to a data storage for a platform (e.g., a network hosted platform, such as cloud system 106, as discussed supra) or a plurality of platforms. Database 108 may receive storage instructions/requests from, for example, compliance engine 200 (and associated microservices), which may be in any type of known or to be known format, such as, for example, standard query language (SQL). According to some embodiments, database 108 may correspond to any type of known or to be known storage, for example, a memory or memory stack of a device, a distributed ledger of a distributed network (e.g., blockchain, for example), a look-up table (LUT), and/or any other type of secure data repository.

Compliance engine 200, as discussed above and further below in more detail, can include components for the disclosed medical monitoring functionality. According to some embodiments, compliance engine 200 may be a special purpose machine or processor, and can be hosted by a device on network 104, within cloud system 106, on AP device 112 and/or on UE 102. In some embodiments, compliance engine 200 may be hosted by a server and/or set of servers associated with cloud system 106.

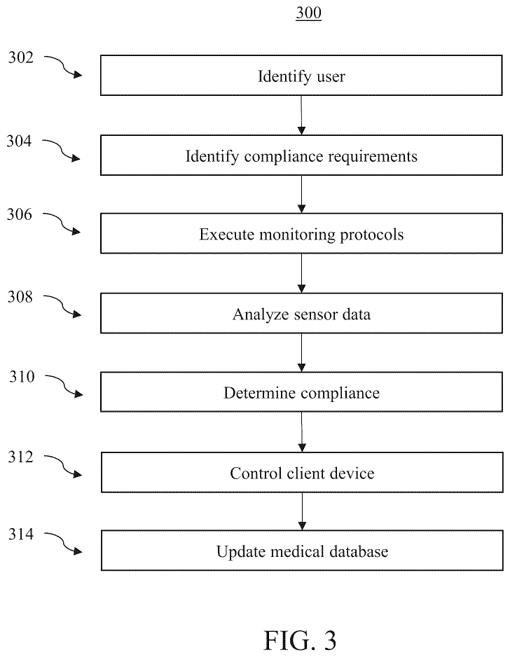

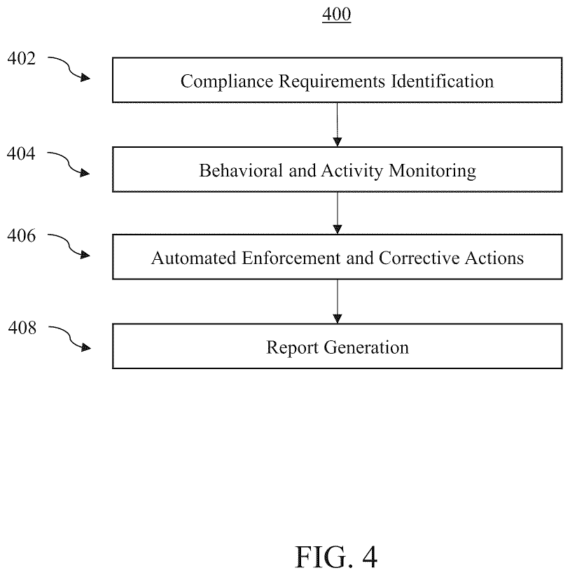

According to some embodiments, as discussed in more detail below, compliance engine 200 may be configured to implement and/or control a plurality of services and/or microservices, where each of the plurality of services/microservices are configured to execute a plurality of workflows associated with performing the disclosed application control and management framework. Non-limiting embodiments of such workflows are provided below in relation to at least FIGS. 3-4.

According to some embodiments, as discussed above, compliance engine 200 may function as an application provided by cloud system 106. In some embodiments, compliance engine 200 may function as an application installed on a server(s), network location and/or other type of network resource associated with system 106. In some embodiments, compliance engine 200 may function as application installed and/or executing on UE 102 (and/or AP device 112, in some embodiments). In some embodiments, such application may be a web-based application accessed by AP device 112 and/or UE over network 104 from cloud system 106. In some embodiments, compliance engine 200 may be configured and/or installed as an augmenting script, program or application (e.g., a plug-in or extension) to another application or program provided by cloud system 106 and/or executing on AP device 112 and/or UE 102.

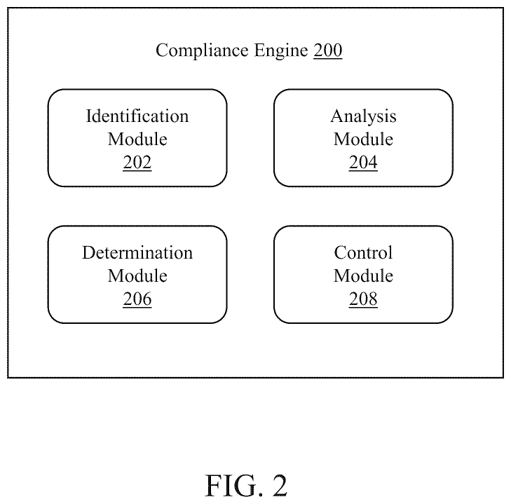

As illustrated in FIG. 2, according to some embodiments, compliance engine 200 includes identification module 202, analysis module 204, determination module 206, and control module 208. It should be understood that the engine(s) and modules discussed herein are non-exhaustive, as additional or fewer engines and/or modules (or sub-modules) may be applicable to the embodiments of the systems and methods discussed.

More detail of the operations, configurations and functionalities of compliance engine 200 and each of its modules, and their role within embodiments of the present disclosure will be discussed below. For example, as further detailed in FIGS. 8-14, compliance engine 200 may include submodules 801-807, and/or any other submodules described herein, such as those described in relation to real-time personalization 1201. As further detailed in FIGS. 8-14, identification module 202 may be surfaced via interactive patient interface 806 and data ingestion module 802; analysis module 204 may consume interaction capture 1205 and sensor inputs; determination module 206 (including safety module 805) adjudicates compliance; and control module 208 issues actions through adaptive content engine 804 and interactive patient interface 806.

Turning to FIG. 3, process 300 provides non-limiting example embodiments for the disclosed medical compliance software. According to some embodiments, Process 300 provides an illustration of steps for which the disclosed framework (e.g., via compliance engine 200) can control, manage, and manipulate the UE 102. Steps described in the figures represent both an execution of a computer algorithm and a method of implementing the system in accordance with some embodiments.

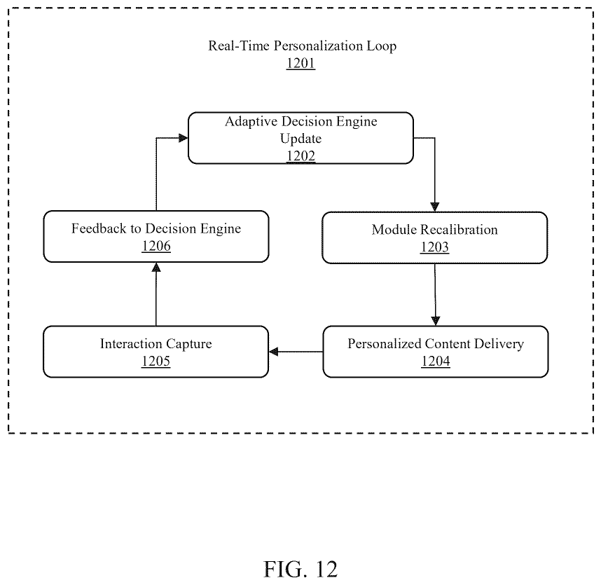

According to some embodiments, steps 302-304 of process 300 can be performed by identification module 202 of compliance engine 200; steps 306 and 312 can be performed by control module 208; and steps 308 can be performed by analysis module 204; and steps 310 and 314 can be executed by determination module 206. As further detailed in FIG. 12, real-time personalization loop 1201 operationalizes process 300 end-to-end using data ingestion module 802, adaptive decision engine 803, adaptive content engine 804, safety module 805, interactive patient interface 806, and documentation and coding engine 807 (EMR output 808).

It should be understood that while the discussion herein will be with reference to the disclosed framework, it should not be construed as limiting, as any type of program, website, network resource, platform or device (e.g., any of the UEs discussed above) can be used as a portion of the medical compliance monitoring system without departing from the scope of the instant disclosure.

According to some embodiments, process 300 begins with step 302 where engine 200 is configured to identify the patient (user). According to some embodiments, the process of patient identification can begin with basic username and password authentication, where users provide a unique username and corresponding password to verify their identity. In some embodiments, multi-factor authentication (MFA) is employed, incorporating multiple forms of authentication, such as a password, a code, and biometric verification (e.g., fingerprint).

In some embodiments, the system is configured to request fingerprint identification. Fingerprint recognition involves users authenticating themselves with scanners that match their fingerprints against stored data. In some embodiments, the system is configured to execute biometric identification that includes an AI-based facial recognition platform that match a user's face against a pre-registered database using real-time camera images. In some embodiments, the system is configured to execute iris/retina scanning that uses imaging techniques to identify unique patterns to identify a user. In some embodiments, the system is configured to execute voice recognition to authenticate users based on vocal patterns.

In some embodiments, GPS or geofencing is used by the system, where the system is configured to track user location (e.g., via mobile devices), ensuring compliance-critical tasks are conducted in authorized areas, such as a hospital waiting room. In some embodiments, Wi-Fi/Bluetooth tracking executed by the system ensures user proximity to specific areas by monitoring network connections.

In some embodiments, the system is configured to use data from wearable devices to identify a patient. For example, smartwatches and fitness trackers equipped with biometric sensors can provide physiological data to verify identity. In some embodiments, the system includes body-worn sensors with environmental or physiological tracking capabilities that are configured for continuous identification.

In some embodiments, continuous monitoring for identification involves real-time presence tracking using multiple sensors to verify location and compliance during tasks, such as when watching educational modules such as pre-operative and/or procedural videos and/or simulations. Mobile device biometrics, like FaceID® or fingerprint scans, enable identity verification via smartphones, with AI systems processing this data for security in accordance with some embodiments.

In some embodiments, multiple identification methods may be combined for enhanced security. As a non-limiting example, in some embodiments, facial recognition is used alongside behavioral monitoring to ensure compliance with both identification verification and comprehension tracking, reflecting the improvement in the art by the executing novel combinations of different platforms within the identification framework.

In step 304, compliance engine 200 is configured to identify compliance requirements in accordance with some embodiments. As a non-limiting example, compliance engine 200 can access a medical database to determine a type of patient understanding and compliance needed for a particular procedure, as different medical procedures pose different risks to the patient and/or the surgeon. Advantageously, in some embodiments, the system can automatically configure one or more sensors (e.g., cameras, microphones) and/or program execution steps based on one or more of the type of procedure, the demographic of the patient, the jurisdiction of the procedure, the requirements of an insurance provider, required documentation, and/or any user defined parameters.

In step 306, the system is configured to execute monitoring protocols in accordance with some embodiments. Various monitoring protocols can be implemented independently and/or simultaneously, where non-limiting examples include camera-based eye tracking, facial recognition and presence detection, infrared sensors monitoring head and/or body position, motion detection, proximity detection, keystroke and/or display interaction, audio monitoring, and/or posture monitoring.

At step 308, the data collected through one or more sensors is analyzed in accordance with some embodiments. In some embodiments, the system includes one or more cameras configured to identify whether a user is present by recognizing their face, ensuring the right person is participating and actively involved. In some embodiments, the system uses one or more cameras to detect body movements or gestures to confirm that the user remains in front of the information display. In some embodiments, the system is configured to compare a patient's field of vision to a location of the information presentation.

In some embodiments, the system includes analysis algorithms that calculate the gaze coordinates and determine whether the user is looking at the relevant screen area. In some embodiments, by analyzing the duration and frequency of gaze fixations, the system assesses the user's attention span, where if the gaze frequently shifts away from the screen, it might signal disengagement. In some embodiments, the system is configured to monitor blink rates to determine fatigue or focus. A high blink rate may indicate that the user is distracted or tired, which can affect engagement.

At step 310, the system is configured to determine compliance by comparing an analysis result for one or more monitored parameter to one or more compliance threshold values in accordance with some embodiments. In some embodiments, the system is able to save computer resources by only implementing monitoring protocols determined at step 304.

At step 312, in some embodiments, the system is configured to control one or more client devices, such as UE 102, when noncompliance is detected. In some embodiments, the system offers a dynamic compliance monitoring framework by controlling the UE 102 in real-time when one or more monitored parameters do not meet a predetermined threshold. In some embodiments, the system is configured to control one or more of: video speed (e.g., play/pause), chat bot engagement, notifications, and/or repetition of specific segments when the user does not pass compliance thresholds, as non-limiting examples.

In some embodiments, the system is configured to cause a message or pop-up to appear on the display, reminding the user to stay focused and/or asking if they are still following along. In some embodiments, the system can present interactive activities to the user (e.g., patient) when the system detects a drop in engagement, encouraging users to interact and re-engage with the content. In some embodiments, the system is configured to flag sections of content that were displayed when the user was not paying attention and highlight these for review once they re-engage. In some embodiments, the system requires the user to perform a manual action, such as pressing a key, clicking a button, or answering a question, to confirm they are still engaged. In some embodiments, a voice over is used to describe portions of a display. In some embodiments, an avatar is used in conjunction with, or in place of, a voice over.

In some embodiments, the system automatically pauses the content whenever the user's gaze moves away from the screen. When the user refocuses on the content, the system is configured to resume without requiring manual interaction in accordance with some embodiments. In some embodiments, eye tracking is used by the system to trigger actions directly based on where the user is looking. For example, if the user is repeatedly looking away, the system is configured to prompt them with interactive elements or ask if they want to continue the current session.

At step 314, the system is configured to update one or more portions of a medical database. In some embodiments, the system is configured to access, review, and or complete one or more consent forms in the medical database. In some embodiments, one or more consent forms include the nature of the medical procedure, risks, benefits, and/or alternatives, ensuring that the patient is fully informed before proceeding. In some embodiments, the system is configured to ensure that the patient voluntarily agrees to the procedure after receiving clear, understandable information by monitoring biometrics such as heart rate, facial expression, subject matter focus, and/or voice analysis, as non-limiting examples.

In some embodiments, the system is configured to access, review, and/or complete one or more preoperative checklists and detailed records of the procedure. In some embodiments, system is configured to use one or more sensors, biometrics, and/or patient monitoring to ensure the correct patient, procedure, and/or surgical site is associated with the correct individual, ensuring compliance with safety protocols.

Medical procedures, treatments, and patient records must comply with data protection laws like HIPAA. In some embodiments, the system is configured to limit exposure to patient data by automatically filling out appropriate portions of the form, ensuring only authorized personnel have viewed or edited the patient's medical history. In some embodiments, the system is configured to add a list of all current medications to medical procedure forms for the individual identified by the various configurations described herein. In some embodiments, the system is configured to ensure compliance with postoperative instructions and/or automatically fill out documents based on observed compliance to ensure compliance with aftercare protocols, patient safety, and regulatory guidelines.

In some embodiments, the system is configured to automatically complete and/or verify documents related to billing and insurance. In some embodiments, the documents include compliance with insurer requirements for pre-authorization, ensuring that the procedure is covered. In some embodiments, the system is configured to verify billing codes (ICD, CPT) comply with healthcare reimbursement standards.

As discussed previously different AI models may be used independently and/or in conjunction with one or more other steps, such as those described in Process 300 and Process 400.

Turning now to FIG. 4, a non-limiting example Process 400 that includes the execution of one or more AI models. At step 402, one or more AI models are used to determine compliance requirements. In some embodiments, the system is configured to execute one or more natural language processing (NLP) models for interpreting, extracting, and organizing information from one or more databases. NLP models are configured to use techniques to understand and process human language, making them suitable for scanning large datasets and extracting relevant compliance-related details. In some embodiments, the system includes one or more Transformer-based Models (e.g., Bidirectional Encoder Representations from Transformers (BERT), Generative Pretrained Transformer (GPT)) configured to read and understand medical records, policies, and regulatory documents. In some embodiments, the AI models are configured to search through databases to find specific phrases, conditions, or codes related to compliance, and organize the findings into a list. In some embodiments, the system includes one or more Named Entity Recognition (NER) models configured for identifying and extracting key entities from text, such as dates, patient names, procedure codes, medications, and compliance-related terms (e.g., “HIPAA,” “FDA guidelines”). NER models help ensure that relevant compliance information is retrieved accurately in accordance with some embodiments.

In some embodiments, the system is configured to execute traditional rule-based systems in conjunction with one or more AI models for tasks where strict adherence to regulations is essential. In some embodiments, the one or more AI models extract relevant data, while the rule-based component checks the data against compliance guidelines. In some embodiments, the system is configured to execute a hybrid model to process claims, extract relevant procedure codes using NLP, and/or check the codes against a rule-based system that determines which procedures are covered by the policy.

At step 404, one or more AI models are used for behavioral and activity monitoring. In some embodiments, the system can use various sensors (such as cameras, eye trackers, or motion sensors) in conjunction with AI to understand how users interact with content and detect when they disengage. For example, in some embodiments, the system is configured to use one or more Convolutional Neural Networks (CNNs) to analyze live video feeds to track facial expressions and head movements, determining whether a user is paying attention. In some embodiments, Recurrent Neural Networks (RNNs) are used to process sequences of gaze or motion data to predict disengagement over time. In some embodiments, Support Vector Machines (SVMs) are configured to classify user behaviors, such as “engaged” or “disengaged,” based on multi-sensor data input.

In some embodiments, the system is configured to execute pose estimation algorithms to determine the orientation of the user's head and face. In some embodiments, if the face is not oriented towards the screen, the system is configured to flag the user as inattentive or absent.

Some embodiment execute one or more AI models to analyze the angles and distances of the head and body from the screen. If the user's position is too far or not aligned with the screen (indicating they are turned away), the system identifies this as disengagement, and execute one or more control commands as described below. In some embodiments, the system defines a threshold distance from the screen and triggers alerts if the user moves beyond this range, indicating they are no longer interacting with the device. In some embodiments, motion sensors track small user movements (like hand or body adjustments) to monitor presence and/or awareness. Lack of motion over a specified period signals that the user might have left or is no longer engaged, leading the system to conclude inactivity and take one or more actions described below.

In some embodiments, one or more AI models analyze typing speed, rhythm, and/or patterns to determine if the user is actively interacting with the system. Deviations in typical typing patterns can indicate inattentiveness or distraction. In some embodiments, the system tracks mouse movements, clicks, and/or hover patterns. If there is a lack of activity (e.g., no mouse movement or clicks for a certain time), the system concludes the user as potentially disengaged in accordance with some embodiments.

In some embodiments, the system detects speech using Voice Activity Detection (VAD) algorithms, which analyze the audio signal for speech patterns. If no speech is detected during expected periods (e.g., during a video call), the system may conclude the user is inactive. In some embodiments, machine learning models are executed to classify ambient sounds (e.g., background noise). In some embodiments, if excessive background noise is detected, the system can infer that the user may be distracted by their surroundings.

In some embodiments, the system is configured to merge data from multiple sensors (eye-tracking, motion sensors, facial recognition) to create a comprehensive profile of user engagement. This improves accuracy by cross-referencing data from different sources. In some embodiments, AI models are configured to calculate an engagement score based on sensor inputs, where a combination of factors such as continuous gaze on the screen, facial presence, and active body posture indicate high engagement. Low engagement is flagged when multiple signals suggest inattention (e.g., gaze off-screen, no movement, slouching) in accordance with some embodiments.

At step 406, the one or more AI models are used for automated enforcement and/or corrective actions. AI activity modeling can dynamically respond to user behavior in real-time. If a system detects that a user is losing focus, it might pause content or trigger engagement-boosting actions like quizzes or pop-ups in accordance with some embodiments. In some embodiments, the system is configured to execute Reinforcement Learning (RL) to adjust content delivery based on feedback from the user's responses to previous actions. In some embodiments, the RL model is configured to deploy a pop-up reminder when engagement drops, or offer alternative content formats if users consistently disengage. In some embodiments, the system is configured to execute decision trees to evaluate various engagement-related metrics (e.g., gaze, activity levels) and decide whether to pause or continue the content. In some embodiments, Gradient Boosting Machines (GBMs) are employed to make more granular predictions about which engagement strategies work best for different types of users.

At step 408, the system uses one or more AI models to generate one or more reports. In some embodiments, the system is configured to generate a detailed audit report that documents user interactions and compliance status. In some embodiments, the report includes timestamps, user IDs, activities performed, engagement metrics, and/or any deviations from expected behavior. In some embodiments, each interaction is logged, allowing for comprehensive tracking of user actions over time. In some embodiments, the system includes one or more Natural Language Generation (NLG) Models to automatically generate written summaries and insights from the analyzed data. In some embodiments, the system uses one or more AI models to analyze user interactions and determine where content may be improved to enhance user engagement. For example, if most users pay less attention to certain segments, those segments can be flagged for further analytics and potential revision.

In some embodiments, such computational analysis can involve compliance engine 200 executing any type of computational analysis technique, algorithm, mechanism or technology. In some embodiments, compliance engine 200 may include a specific trained artificial intelligence model (AI), such as a particular machine learning model architecture, a particular machine learning model type (e.g., convolutional neural network (CNN), recurrent neural network (RNN), autoencoder, support vector machine (SVM), and the like), or any combination of models discussed herein.

In some embodiments, compliance engine 200 may be configured to utilize one or more AI techniques chosen from, but not limited to, computer vision, feature vector analysis, decision trees, boosting, support-vector machines, neural networks, nearest neighbor algorithms, Naïve Bayes, bagging, random forests, logistic regression, and the like. By way of a non-limiting example, compliance engine 200 can implement an XGBoost algorithm for regression and/or classification to analyze the sensor data, as further discussed infra.

In some embodiments and, optionally, in combination of any embodiment described above or below, an AI neural network technique may be one of, without limitation, feedforward neural network, radial basis function network, recurrent neural network, convolutional network (e.g., U-net) or other suitable network. In some embodiments and, optionally, in combination of any embodiment described above or below, an implementation of neural network may be executed as follows:
a. define neural network architecture/model for the control framework,
b. transfer the input data to the neural network model,
c. train the model incrementally,
d. determine the accuracy for a specific number of timesteps,
e. apply the trained model to process the newly received input data,
f. optionally and in parallel, continue to train the trained model with a predetermined periodicity.

In some embodiments and, optionally, in combination of any embodiment described above or below, the trained AI model may specify a neural network by at least a neural network topology, a series of activation functions, and connection weights. For example, the topology of a neural network may include a configuration of nodes of the neural network and connections between such nodes. In some embodiments and, optionally, in combination of any embodiment described above or below, the trained AI model may also be specified to include other parameters, including but not limited to, bias values/functions and/or aggregation functions. For example, an activation function of a node may be a step function, sine function, continuous or piecewise linear function, sigmoid function, hyperbolic tangent function, or other type of mathematical function that represents a threshold at which the node is activated. In some embodiments and, optionally, in combination of any embodiment described above or below, the aggregation function may be a mathematical function that combines (e.g., sum, product, and the like) input signals to the node. In some embodiments and, optionally, in combination of any embodiment described above or below, an output of the aggregation function may be used as input to the activation function. In some embodiments and, optionally, in combination of any embodiment described above or below, the bias may be a constant value or function that may be used by the aggregation function and/or the activation function to make the node more or less likely to be activated.

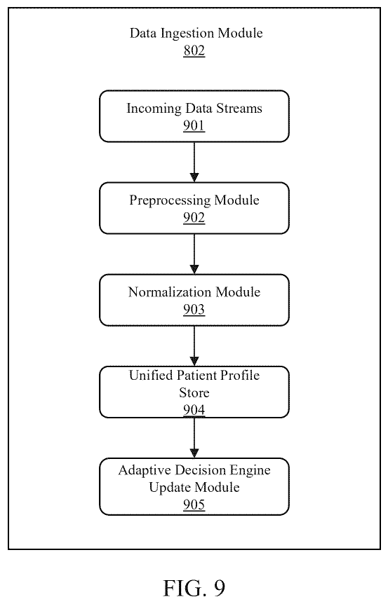

As further detailed in FIGS. 9-11 and 14, one or more AI models referenced herein are executed within data ingestion module 802 (including NLP/NER), adaptive decision engine 803 (risk, learning, temporal models), adaptive content engine 804 (branching and language adaptation), and documentation and coding engine 807 (summarization and coding extraction), and are orchestrated by the loop in FIG. 12.

FIG. 7 is a schematic diagram illustrating a client device showing an example embodiment of a client device that may be used within the present disclosure and/or the framework illustrated in FIG. 1. Client device 700 may include many more or less components than those shown in FIG. 7, such as a plurality of one or more computers. However, the components shown are sufficient to disclose an illustrative embodiment for implementing the present disclosure. Client device 700 may represent, for example, UE 102 discussed above at least in relation to FIG. 1.

As shown in the figure, in some embodiments, client device 700 includes one or more processors (CPU) 722 in communication with one or more non-transitory computer readable media 730 via a bus 724. Client device 700 also includes a power supply 726, one or more network interfaces 750, an audio interface 752, a display 754, a keypad 756, an illuminator 758, an input/output interface 760, a haptic interface 762, an optional global positioning systems (GPS) receiver 764 and a camera(s) or other optical, thermal or electromagnetic sensors 766. Device 700 can include one camera/sensor 766, or a plurality of cameras/sensors 766, as understood by those of skill in the art. Power supply 726 provides power to Client device 700.

Client device 700 may optionally communicate with a base station (not shown), or directly with another computing device. In some embodiments, network interface 750 is sometimes known as a transceiver, transceiving device, or network interface card (NIC).

Audio interface 752 is arranged to produce and receive audio signals such as the sound of a human voice in some embodiments. Display 754 may be a liquid crystal display (LCD), gas plasma, light emitting diode (LED), or any other type of display used with a computing device. Display 754 may also include a touch sensitive screen arranged to receive input from an object such as a stylus or a digit from a human hand.

Keypad 756 may include any input device arranged to receive input from a user. Illuminator 758 may provide a status indication and/or provide light.

Client device 700 also includes input/output interface 760 for communicating with external. Input/output interface 760 can utilize one or more communication technologies, such as USB, infrared, Bluetooth™, or the like in some embodiments. Haptic interface 762 is arranged to provide tactile feedback to a user of the client device.

Optional GPS transceiver 764 can determine the physical coordinates of Client device 700 on the surface of the Earth, which typically outputs a location as latitude and longitude values. GPS transceiver 764 can also employ other geo-positioning mechanisms, including, but not limited to, triangulation, assisted GPS (AGPS), E-OTD, CI, SAI, ETA, BSS or the like, to further determine the physical location of client device 700 on the surface of the Earth. In one embodiment, however, Client device may, through other components, provide other information that may be employed to determine a physical location of the device, including for example, a MAC address, Internet Protocol (IP) address, or the like.

Mass memory 730 includes a RAM 732, a ROM 734, and other storage means. Mass memory 730 illustrates another example of computer storage media for storage of information such as computer readable instructions, data structures, program modules, usage data, or other data. Mass memory 730 stores a basic input/output system (“BIOS”) 740 for controlling low-level operation of Client device 700. The mass memory also stores an operating system 741 for controlling the operation of Client device 700.

Memory 730 further includes one or more data stores, which can be utilized by Client device 700 to store, among other things, applications 742 and/or other information or data. For example, data stores may be employed to store information that describes various capabilities of Client device 700. The information may then be provided to another device based on any of a variety of events, including being sent as part of a header (e.g., index file of the HLS stream) during a communication, sent upon request, or the like. At least a portion of the capability information may also be stored on a disk drive or other storage medium (not shown) within Client device 700.

Applications 742 may include computer executable instructions which, when executed by Client device 700, transmit, receive, and/or otherwise process audio, video, images, and enable telecommunication with a server and/or another user of another client device. Applications 742 may further include a client that is configured to send, to receive, and/or to otherwise process gaming, goods/services and/or other forms of data, messages and content hosted and provided by the platform associated with engine 200 and its affiliates.

In some embodiments, the system is configured to link with one or more Electronic Medical Record (EMR) databases and/or directly access and exchange patient information with an EMR platform. This eliminates redundant data entry, ensures data consistency, and enhances clinical efficiency by automating workflows and reducing manual errors.

In some embodiments, sensor(s) 766 may include various devices that provide multimodal inputs used for identification, presence verification, engagement monitoring, and personalization. Non-limiting sensor modalities include RGB and infrared (IR) cameras for face and gaze estimation; depth cameras; inertial measurement units (IMU) and accelerometers for motion and posture; proximity and ambient-light sensors; microphones for voice-activity detection (VAD) and ambient-noise classification; Bluetooth Low Energy (BLE) beacons and Wi-Fi/Bluetooth signal strength for proximity and geofencing; and external peripherals such as wearables that provide physiologic signals (e.g., heart rate, blood pressure, temperature, and ambulation metrics).

In some embodiments, sensor(s) 766 may be embedded in UE 102 (e.g., smartphone or tablet sensors) or connected as peripherals via known pairing mechanisms (e.g., Wi-Fi, BLE, NFC, USB). In some embodiments, interactive patient interface 806 orchestrates sensor capture on the endpoint and performs lightweight preprocessing (e.g., frame sampling, audio segmentation, feature extraction) with optional on-device inference for low-latency tasks (e.g., presence/gaze checks), and streams structured signals to data ingestion module 802 and adaptive decision engine 803 for real-time decisions.

In some embodiments, sensor fusion combines multiple modalities to compute engagement metrics (e.g., face present, head orientation, gaze coordinates, blink rate, posture, distance to display, ambient noise), which determination module 206 (including safety module 805) evaluates against thresholds to trigger control actions. In some embodiments, privacy controls limit storage to derived metrics and timestamps; raw frames/audio may be processed ephemerally on the device with encryption in transit and at rest, and with role-based access controls and audit logging applied to any persisted data. In some embodiments, the system handles occlusion, low-light, and off-angle conditions by confidence scoring and fallback to interaction-based signals (e.g., keyboard/mouse/touch events) to avoid false noncompliance determinations. As further detailed in FIGS. 9-12, sensor-derived signals enter the ingestion and personalization pipeline and drive adaptive content adjustments and documentation.

FIG. 8 illustrates a non-limiting system execution workflow for submodules of compliance engine 200, in accordance with some embodiments. In some embodiments, compliance engine 200 is configured to ingest electronic medical record (EMR) data and events, perform adaptive decisioning, validate generated content under clinical guardrails, deliver personalized patient education through interactive endpoints, capture patient interactions, and automatically generate EMR-ready documentation. In some embodiments, one or more EMR platforms 801 communicate with compliance engine 200 via healthcare data exchange standards (e.g., HL7, FHIR) and application programming interfaces (APIs), enabling continuous event monitoring for diagnoses, laboratory results, imaging findings, medications, vital signs, clinical notes, and scheduling updates. In some embodiments, incoming clinical and operational signals flow into a data ingestion module 802 that serves as the gateway for structured and unstructured inputs and real-time events and that produces normalized and profiled outputs for downstream components, as further detailed in FIG. 9.

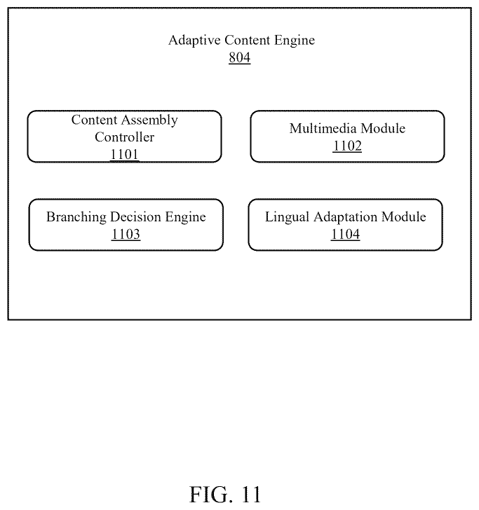

Turning back to FIG. 8, in some embodiments, an adaptive decision engine 803 (also referred to herein as a “digital twin”) operates as a subsystem of compliance engine 200 and maintains a patient-specific decision state that is updated in real time based on normalized inputs from data ingestion module 802. In some embodiments, adaptive decision engine 803 orchestrates downstream content selection, risk emphasis, sequencing, and alerting behaviors that are consumed by an adaptive content engine 804, which assembles multimodal, multilingual, and literacy-adapted educational modules tailored to the current clinical context and patient profile as further described in relation to FIG. 11.

In some embodiments, determination module 206 includes a safety module 805 (submodule). In some embodiments, content generated by adaptive content engine 804 is vetted by a safety module 805 positioned as a gate between content assembly and delivery, where safety module 805 enforces physician-approved templates, institutional guidelines, and clinical/legal guardrails. In some embodiments, safety module 805 applies required phrasing constraints to documentation components generated by a documentation and coding engine 807.

In some embodiments, validated content is delivered to an interactive patient interface 806 that is deployable across one or more computing devices, such as mobile devices, tablets, desktops, and kiosks. In some embodiments, interactive patient interface 806 provides accessibility modes and multilingual support. In some embodiments, the interactive patient interface 806 captures one or more inputs such as quiz responses, symptom inputs, and comprehension confirmations for logging and analysis. In some embodiments, interactive patient interface 806 endpoints operate behind AP device 112 in clinical spaces such as waiting rooms and kiosks, with offline and low-bandwidth support that caches interactions and sensor-derived metrics locally and performs deferred synchronization to data ingestion module 802 when connectivity is restored. In some embodiments, interactive patient interface 806 captures digital consent artifacts, including acknowledgments, initials, and electronic signatures, and associates them with comprehension evidence and required disclosures. In some embodiments, these artifacts are transmitted to documentation and coding engine 807 for compilation into EMR documentation output 808, while safety module 805 enforces required phrasing, mandatory fields, institutional policy checks, and blocks submission until compliance is met.

In some embodiments, documentation and coding engine 807 compiles EMR-ready documentation that summarizes education delivered, comprehension evidence, risks discussed, alternatives presented, medical decision-making (MDM) elements, time-based interactions, and consent-support narratives. In some embodiments documentation and coding engine 807 outputs EMR documentation output 808 as further described in FIG. 14.

In some embodiments, a real-time personalization loop 1201 overlays the architecture and represents a runtime cycle operating across modules 803-806 with feedback returning to adaptive decision engine 803. In some embodiments, documentation and coding engine 807 logs changes and updates, as detailed further in FIG. 12. In some embodiments, provider oversight and customization are integrated via provider-facing views within interactive patient interface 806, which enable clinicians to review and edit content elements and workflow behaviors. In some embodiments provider edits and overrides persist into physician-preference overlays maintained by adaptive decision engine 803 and propagate to adaptive content engine 804, subject to safety module 805 constraints as described in FIG. 13, in accordance with some embodiments.

In some embodiments, predictive analytics embedded in adaptive decision engine 803 adjust content selection and emphasis, trigger alerts, tailor postoperative day-specific instructions, and inform documentation rationale. In some embodiments, risk threshold breaches or contraindications cause escalation to provider views in interactive patient interface 806 for review, intervention, or override.

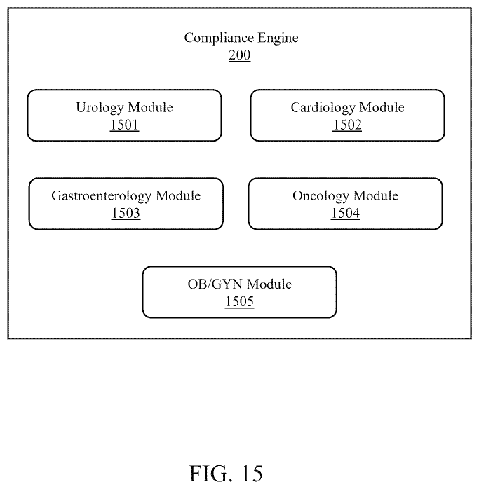

In some embodiments, the architecture supports multi-specialty extensibility using shared core modules 802-807 with specialty-specific content libraries, risk models, templates, and pathway logic layered on top of the core, which are discussed further in relation to FIG. 15. In some embodiments, the system is configured to present recruitment modules and pre consent education for clinical trials through interactive patient interface 806. In some embodiments, the system is configured to deliver standardized information across sites and coordinators and to train research staff on protocol operations using staff facing modules. In some embodiments, the system is configured to track participant progress and to dispatch messages that sustain engagement. In some embodiments, safety module 805 is configured to enforce protocol specific policies and required phrasing. In some embodiments, documentation and coding engine 807 is configured to produce audit ready documentation of participant education and consent for research contexts.

In some embodiments, compliance engine 200 incorporates security and governance aligned with health privacy regulations, including encryption in transit and at rest, role-based access controls, audit logging of protected health information access and safety module actions, provenance tracking from ingestion through decisioning and content delivery, approval workflows for clinician edits, and verification of documentation completeness and legal phrasing prior to EMR insertion.

In some embodiments, the platform is suitable for deployment in infrastructure-as-a-service, platform-as-a-service, and software-as-a-service environments (IaaS/PaaS/SaaS) and scales across enterprise footprints, and in some embodiments multi-agent artificial intelligence components divide responsibilities for content generation, validation, prediction, and documentation assembly under safety module 805 control. In some embodiments, the system supports offline and low-bandwidth operation with caching and deferred synchronization and supports augmentation by internet-of-things (IoT) and wearable devices to incorporate physiologic signals into decisioning and personalization.

FIG. 9 illustrates a process flow for data ingestion module 802, in accordance with some embodiments. In some embodiments, incoming data streams 901 may include structured clinical data received from one or more electronic medical record (EMR) systems, such as International Classification of Diseases version 10 (ICD 10) diagnoses, current procedural terminology (CPT) procedure codes, medication lists, laboratory results, vital signs, imaging results, pathology reports, allergies, problem lists, demographics, and social determinants, as non-limiting examples. In some embodiments, incoming data streams 901 include unstructured clinical data, such as, without limitation, clinical notes, operative reports, referral letters, patient messages, scanned documents, and portable document format (PDF) attachments. In some embodiments, incoming data streams 901 also include appointment and scheduling context, such as visit type, surgeon assignment, urgency indicators, and preparation or medical clearance requirements, as non-limiting examples.

In some embodiments, data ingestion module 802 monitors events from EMR platforms for new or updated records in real time. In some embodiments, data ingestion module 802 queues and prioritizes messages to preserve timeliness for clinically significant updates. In some embodiments, data ingestion module 802 maintains a record of origin for each message to support downstream auditing.

In some embodiments, preprocessing module 902 is configured to perform data cleaning and validation. In some embodiments, preprocessing module 902 performs natural language processing (NLP) and named entity recognition (NER) to extract clinical entities and concepts from unstructured text. In some embodiments, preprocessing module 902 tokenizes free text and maps extracted fields to interim internal keys. In some embodiments, preprocessing module 902 is configured to reject or flag malformed records for review without interrupting the overall pipeline.

In some embodiments, normalization module 903 maps codes and concepts to standardized vocabularies. In some embodiments, normalization module 903 is configured to map to Systematized Nomenclature of Medicine (SNOMED), Logical Observation Identifiers Names and Codes (LOINC), and International Classification of Diseases (ICD) as appropriate. In some embodiments, normalization module 903 harmonizes data into internal schemas suitable for decisioning and content generation. In some embodiments, normalization module 903 records source, timestamp, and confidence metadata for each normalized field.

In some embodiments, database 108 includes a unified patient profile store 904 that persists the normalized outputs. In some embodiments, unified patient profile store 904 maintains clinical parameters, comorbidities, medication state, language and literacy preferences, behavioral and learning profiles, risk categories, specialty pathway assignments, and temporal state indicators for preoperative, perioperative, postoperative, and recovery phases. In some embodiments, unified patient profile store 904 supports incremental updates without full reprocessing. In some embodiments, unified patient profile store 904 exposes change logs and versioning to support audit and rollback.

In some embodiments, adaptive decision engine update module 905 streams deltas from unified patient profile store 904 to adaptive decision engine 803. In some embodiments, decision engine update 905 includes scheduling aware indicators and trigger conditions for initiating or modifying educational modules. In some embodiments, adaptive decision engine update module 905 is configured to distinguish between urgent clinical changes and routine updates. In some embodiments, adaptive decision engine update module 905 tags each change with priority and suggested downstream actions.

In some embodiments, data ingestion module 802 supports integration of signals from internet of things (IoT) and wearable devices. In some embodiments, these signals include heart rate, blood pressure, temperature, and ambulation metrics that augment personalization and trigger loop updates as described in FIG. 12. In some embodiments, data ingestion module 802 supports offline and low bandwidth operation. In some embodiments, the data ingestion module pipeline caches records locally and performs deferred synchronization with conflict resolution upon reconnection.

In some embodiments, security and privacy controls are applied at each stage of FIG. 9, and may include encryption in transit and at rest, role-based access controls, protected health information (PHI) audit logging, and field level provenance. In some embodiments, the data ingestion module pipeline exposes aggregated outputs for quality improvement and operational analytics. In some embodiments, these aggregate views are produced without duplicating patient level logic and are used by dashboard and documentation components described in FIG. 13 and FIG. 14.

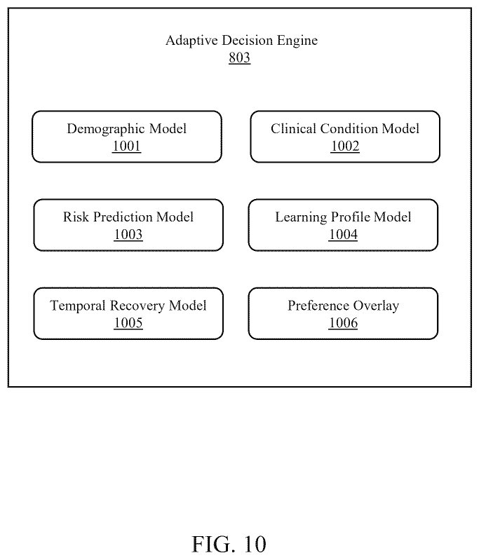

FIG. 10 illustrates internal components of adaptive decision engine 803, in accordance with some embodiments. In some embodiments, adaptive decision engine 803 incorporates continuous learning processes to improve personalization over time. In some embodiments, machine learning and reinforcement learning techniques adjust model parameters based on observed outcomes and engagement, and in some embodiments such updates occur within controlled, auditable workflows that include clinician configurable guardrails, approval steps, versioning, and rollback capabilities to maintain safety and compliance.

In some embodiments, a demographic model 1001 represents patient attributes such as age, sex, language preferences, literacy level, and relevant social context. In some embodiments, demographic model 1001 is configured to weight explanation style, language selection, and risk emphasis to improve comprehension and cultural alignment.

In some embodiments, clinical condition model 1002 represents diagnoses and comorbidities with associated severity and phase. In some embodiments, clinical condition model 1002 is configured to support condition to module matching and pathway selection for educational content.

In some embodiments, a risk prediction model 1003 computes probabilities for clinically relevant outcomes. In some embodiments, non-limiting outcomes include complications, emergency department (ED) visits, hospital readmissions, non-adherence to instructions, appointment cancellations, pain trajectories, and recovery timelines, as non-limiting examples. In some embodiments, risk prediction model 1003 produces statistical confidence scores to guide emphasis and escalation thresholds.

In some embodiments, a learning profile model 1004 captures engagement and comprehension history derived from prior interactions with interactive patient interface 806. In some embodiments, learning profile model 1004 is configured to adapt tone, depth, pacing, and modality and to generate behavior driven predictions to pre-empt comprehension gaps in future content.

In some embodiments, a temporal recovery model 1005 tracks preoperative, perioperative, postoperative, and recovery phases for a given patient. In some embodiments, temporal recovery model 1005 produces time-specific (e.g., per day) expectations and instructions and contributes to dynamic recovery timelines that are reflected in educational modules assembled by adaptive content engine 804.

In some embodiments, a preference overlay 1006 stores physician and institution preferences including tone, risk framing, surgical approach, equipment or technique selections, and template choices. In some embodiments, preference overlay 1006 is configured to modulate decision outputs to reflect clinician configured standards and local policies.

In some embodiments, adaptive decision engine 803 is configured to update internal models and parameters in real time in response to incoming changes. In some embodiments, laboratory result changes adjust risk prediction nodes, imaging findings modify procedure related steps, pathology updates alter prognosis related guidance, medication changes update perioperative instruction logic, new diagnoses initiate relevant educational modules, and scheduling changes adjust urgency and content sequencing, as non-limiting examples.

In some embodiments, adaptive decision engine 803 produces decision outputs that orchestrate downstream content assembly. In some embodiments, the decision outputs include, without limitation, module selection, sequencing, risk emphasis, alternative option presentation, and instruction specificity, and are consumed by adaptive content engine 804 subject to safety module 805 constraints, as further described in FIG. 11.

In some embodiments, adaptive decision engine 803 is configured to issue alerts and triage recommendations to provider facing views within interactive patient interface 806. In some embodiments, alerts are raised when risk thresholds or rule violations are detected, and recommended actions may include notifying staff, scheduling urgent follow ups, generating triage scripts, or temporarily blocking content pending review, as non-limiting examples. In some embodiments, adaptive decision engine 803 logs decision rationale, parameters used, predictions made, and time or context markers to documentation and coding engine 807. In some embodiments, these logs support subsequent documentation generation and audit trail completeness.

FIG. 11 illustrates internal components of adaptive content engine 804, in accordance with some embodiments. In some embodiments, adaptive content engine 804 assembles educational modules that are personalized based on decision outputs received from adaptive decision engine 803. In some embodiments, adaptive content engine 804 is configured to compose multimedia elements, apply interactive branching logic, and adapt language and literacy parameters prior to validation by safety module 805.

In some embodiments, a content assembly controller 1101 orchestrates the composition of educational modules. In some embodiments, content assembly controller 1101 selects topics, orders segments, and applies risk emphasis and instruction specificity according to received directives. In some embodiments, content assembly controller 1101 applies required disclosures and integrates checklists and timelines that correspond to the patient's current clinical phase.

In some embodiments, the system is configured to implement adaptive content module 804. In some embodiments, adaptive content module 804 includes content assembly controller 1101, multimedia module 1102, branching decision module 1103, and lingual adaptation module 1104. In some embodiments, the system is configured to curate a comprehensive eLearn library of medically accurate, patient-friendly educational materials and to present those materials through interactive flows.

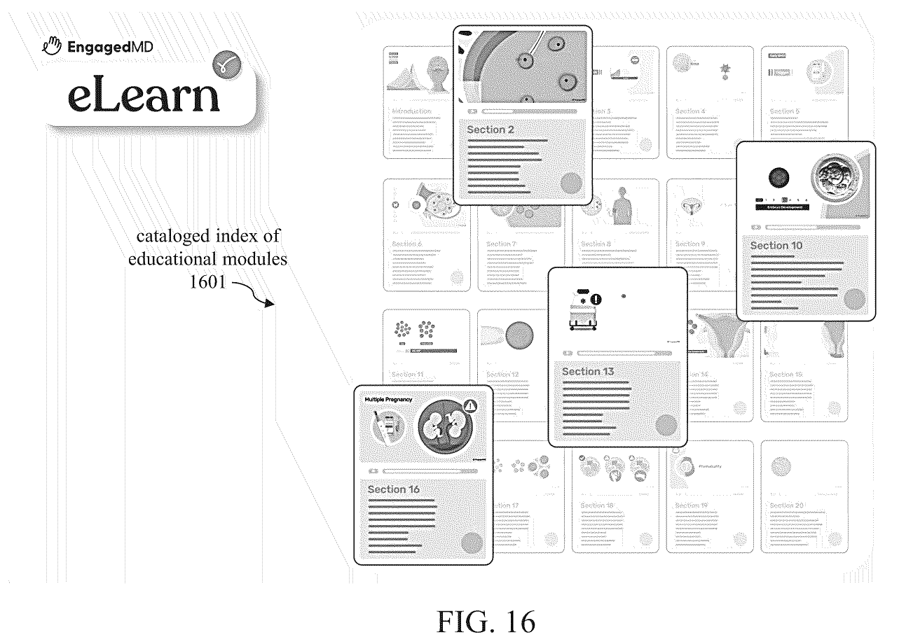

As illustrated in FIG. 16, in some embodiments adaptive content engine 804 is configured to maintain a cataloged index of educational modules 1601 with canonical identifiers, specialty tags, language and literacy metadata, and status attributes for assignment and completion. In some embodiments, provider-facing views within interactive patient interface 806 are configured to render the catalog as selectable module cards with title, thumbnail, summary lines, and visual status indicators corresponding to assignment state and progress. In some embodiments, safety module 805 is configured to enforce institutional governance over catalog visibility and assignment constraints, and documentation and coding engine 807 is configured to persist assignment selections, catalog identifiers, timestamps, and provenance to support audit and EMR documentation output 808.

In some embodiments, the system is configured to embed knowledge checkpoints within modules, to capture responses, and to compute comprehension scores. In some embodiments, the system is further configured to surface comprehension insights to clinical staff for review and follow-up. In some embodiments, the system is configured to track assignment, progress, and completion states for each patient and to determine when reminder messages are due. In some embodiments, the system is configured to dispatch those reminders automatically and to provide video or module replay so patients can revisit key topics.

In some embodiments, the system is configured to personalize protocol-based medication and injection training (e.g., MedReady) to a patient's prescription and clinical protocol. In some embodiments, the system is configured to present stepwise medication instructions and to record training completion for documentation. In some embodiments, the system is configured to assemble and present IV sedation education, including an introduction, risks and potential complications, procedure steps, and pre-sedation preparation. In some embodiments, the system is configured to render subtitles in English and, where available, in additional languages to support accessibility and language preferences. In some embodiments, the system is configured to generate staff-accessible reports of knowledge-checkpoint responses and to provide downloadable files at both the patient level and across cohorts. In some embodiments, the system is configured to define, version, and assign bundles of modules so that related content can be delivered together. In some embodiments, this bundling capability is available even when only eLearn features are enabled. In some embodiments, validated content is delivered by interactive patient interface 806 under clinical and legal guardrails enforced by safety module 805. In some embodiments, documentation and coding engine 807 is configured to log comprehension results, completion events, timestamps, and provenance and to produce EMR-ready outputs.

In some embodiments, multimedia module 1102 generates audiovisual components for delivery. In some embodiments, multimedia module 1102 is configured to render animations, two dimensional and three-dimensional anatomical visualizations, and procedure simulations. In some embodiments, multimedia module 1102 is configured to produce avatar guided explanations with synchronized voice output, and support multilingual voice synthesis using clinically appropriate terminology.

In some embodiments, a branching decision engine 1103 implements interactive pathways within each module. In some embodiments, branching decision engine 1103 presents comprehension checkpoints, symptom input nodes, and preference or risk-based branch selection to tailor the sequence and depth of content. In some embodiments, branching decision engine 1103 is configured to incorporate physician defined decision trees and to invoke escalation paths when concerning inputs are detected by interactive patient interface 806, described further in relation to FIG. 13.

In some embodiments, a lingual adaptation module 1104 localizes content into multiple languages and adjusts reading level and phrasing to match patient literacy. In some embodiments, lingual adaptation module 1104 applies cultural considerations and selects voice personas or text styles that improve clarity and comprehension. In some embodiments, lingual adaptation module 1104 is configured to apply provider or institution preferences received via a preference overlay, as described above in relation to FIG. 10. In some embodiments, adaptive content engine 804 supports accessibility features to improve usability. In some embodiments, accessibility features include large text, high contrast themes, and audio only modes that render uniformly across computing device endpoints via interactive patient interface 806.

In some embodiments, adaptive content engine 804 is configured to assemble consent support elements. In some embodiments, consent support elements include required risk disclosures, alternative options, expected outcomes, and confirmation prompts that demonstrate understanding. In some embodiments, adaptive content engine 804 is configured to compose visit preparation checklists and postoperative recovery timelines aligned to the patient's temporal state and predicted recovery trajectory.

In some embodiments, adaptive content engine 804 executes real time adjustments based on updated decision outputs and interaction signals. In some embodiments, real time adjustments modify module sequence, segment length, tone, risk emphasis, and instruction specificity as patient state and behavior evolve during a session. In some embodiments, adaptive content engine 804 selects additional modules automatically when thresholds or trigger conditions are met.

In some embodiments, (hybrid) safety module 805 validates content assembled by adaptive content engine 804 before delivery. In some embodiments, safety module 805 is configured to enforce physician approved templates, institutional guidelines, and clinical or legal guardrails and may allow, modify, block, or escalate assembled content based on rules or detected risks. In some embodiments, safety module 805 includes additional comprehension checkpoints where indicated by policy.

In some embodiments, adaptive content engine 804 logs content delivery metadata for documentation. In some embodiments, logged metadata include modules and topics delivered, comprehension checkpoint results, risk explanations presented, alternatives discussed, and time on task. In some embodiments, logged metadata are provided to documentation and coding engine 807 for use in generating medical decision making and time-based documentation.

In some embodiments, adaptive content engine 804 supports optional immersive delivery modes. In some embodiments, immersive delivery modes include virtual reality and augmented reality presentations of procedures or recovery activities that are governed by content assembly controller 1101 and branching decision engine 1103. In some embodiments, adaptive content engine 804 may utilize multi-agent artificial intelligence components to generate patient friendly explanations, validate clinical statements, assemble media assets, and prepare documentation snippets, that are monitored by safety module 805.

FIG. 12 illustrates a real-time personalization loop 1201 pipeline for compliance engine 200, in accordance with some embodiments. In some embodiments, processes 300 and 400 are operationalized by real-time personalization loop 1201 as follows: step 302 (identify user) is performed via authentication and identity capture at interactive patient interface 806 with signals persisted via data ingestion module 802; step 304 (identify compliance requirements) is performed by adaptive decision engine 803 using normalized EMR inputs from data ingestion module 802 and NLP/NER models described in process 400 step 402; steps 306 and 308 (execute monitoring protocols and analyze sensor data) correspond to interaction capture 1205 and analysis by analysis module 204; step 310 (determine compliance) is performed by determination module 206, which includes safety module 805; step 312 (control client device) is executed through module recalibration 1203 and personalized content delivery 1204 via adaptive content engine 804 and interactive patient interface 806; and step 314 (update medical database) is performed by documentation and coding engine 807, which produces EMR documentation output 808.

In some embodiments, the loop is initiated by electronic medical record (EMR) events received via data ingestion module 802 and by patient-initiated signals captured through interactive patient interface 806. In some embodiments, example EMR events may include new or updated diagnoses, laboratory results, imaging findings, pathology reports, medications, vital signs, clinical notes, and scheduling updates. In some embodiments, non-limiting example patient-initiated signals may include symptom reports, comprehension failures, and interaction patterns observed during active module delivery.

In some embodiments, the system is configured to execute an adaptive decision engine update 1202 that recalculates patient-specific parameters, risk predictions, and temporal phase indicators using normalized changes received from adaptive decision engine update module 905. In some embodiments, adaptive decision engine update 1202 applies physician preference overlay settings and produces confidence scores that guide emphasis and potential escalation.

In some embodiments, a module recalibration 1203 execution adjusts educational module selection, sequencing, segment length, tone, risk emphasis, alternatives, and instruction specificity based on the updated decision state. In some embodiments, the module recalibration 1203 is scheduling-aware and intensifies preparation content and reminders as procedure dates approach.

In some embodiments, the system is configured to evaluate scheduling signals and assign educational modules at appropriate points along a patient's journey. In some embodiments, the system is configured to determine reminder cadences and to dispatch reminders automatically when deadlines approach. In some embodiments, the system is configured to intensify preparation content as a procedure date nears and to de-emphasize content once comprehension and completion are demonstrated. In some embodiments, the system is configured to deliver recalibrated modules across endpoints through interactive patient interface 806, including mobile devices, tablets, desktops, and kiosks. In some embodiments, the system is configured to capture interactions and comprehension results during delivery and to write structured logs to documentation and coding engine 807. In some embodiments, the system is configured to update decision parameters based on newly captured signals and to adjust subsequent module selection and sequencing accordingly. In some embodiments, the system is configured to enforce clinical and legal guardrails by safety module 805 and to insert mandatory checkpoints where required by policy or risk thresholds.

In some embodiments, the safety module 805 is configured to validate recalibrated content prior to delivery and enforce physician-approved templates, institutional guidelines, and clinical or legal guardrails. In some embodiments, safety module 805 may allow, modify, block, or escalate content based on rules or detected risks, and may require additional comprehension checkpoints where indicated by policy.

In some embodiments, a personalized content delivery 1204 implementation includes sending validated content to interactive patient interface 806 with minimal session disruption, rendering uniformly across mobile, tablet, desktop, and kiosk endpoints, while supporting accessibility and multilingual features.

In some embodiments, an interaction capture 1205 execution records patient quiz results, branching choices, symptom inputs, and engagement or comprehension metrics during delivery. In some embodiments, interaction capture 1205 includes writing structured logs to documentation and coding engine 807 for subsequent medical decision-making and time-based documentation, as further described in relation to FIG. 14. In some embodiments, the real-time personalization loop 1201 operationalizes step 312 (client device control) by instructing adaptive content engine 804 and interactive patient interface 806 to pause, prompt, repeat, or escalate when analysis module 204 and decision engine 803 detect noncompliance.

In some embodiments, a feedback to adaptive decision engine 1206 step includes updating one or more of the learning profile, temporal recovery, risk prediction, and preference overlay models to refine subsequent content and guidance. In some embodiments, feedback to adaptive decision engine 1206 supports continuous personalization across loop iterations and informs escalation criteria for provider-facing views.

In some embodiments, the loop triggers escalation when risk thresholds or rule violations are detected by adaptive decision engine 803 and/or safety module 805. In some embodiments, escalation actions may include notifying staff, proposing urgent follow-ups, generating triage scripts, or temporarily blocking content pending review within provider-facing views, as non-limiting examples.

In some embodiments, the loop supports automated patient messaging and follow-up cadence, which may be vetted by safety module 805 before dispatch. In some embodiments, the automated messaging is configured to adapt content frequency, channel, and emphasis based on risk predictions and interaction history.

In some embodiments, wearable and internet-of-things (IoT) signals are incorporated into loop triggers and personalization. In some embodiments, non-limiting wearable and IoT inputs include heart rate, blood pressure, temperature, ambulation metrics, and respective devices, that are used to adjust content and alert thresholds. In some embodiments, offline and low-bandwidth operation is supported by caching recalibrated content and deferring synchronization of logs and model updates until connectivity is restored. In some embodiments, the loop performs conflict resolution on reconnect to preserve data integrity and auditability.

FIG. 13 illustrates provider-facing views within interactive patient interface 806, including structured clinical questioning and symptom intake, in accordance with some embodiments. In some embodiments, interactive patient interface 806 is configured to display a patient list panel 1301 for clinician oversight. In some embodiments, patient list panel 1301 is configured to display one or more of module completion status, engagement and comprehension scores, pending items, risk flags, and alert prioritization derived from adaptive decision engine 803 outputs and/or documentation and coding engine 807 logs. In some embodiments, patient list panel 1301 enables filtering and sorting to surface time-sensitive or high-risk cases.

In some embodiments, the system is configured to provide assignment management views within interactive patient interface 806. In some embodiments, the system is configured to present an “Assigned By” filter and to improve search and sorting on an assignment overview page (e.g., Episode page), including sortable columns for assignment name, due date, status, assigned on, and assigned by. In some embodiments, the system is configured to provide filters for forms and modules on the assignment overview page to narrow results.

In some embodiments, the system is configured to render a patient directory page (e.g., All Patients page) sortable and searchable by patient name, email, and date created, and searchable across patient name, patient ID, and email. In some embodiments, the system is configured to make entries in a staff task list (e.g., My Tasks) clickable so that staff can initiate actions such as sign, remind, download, void, or refresh directly from the list.

In some embodiments, interactive patient interface 806 may normalize email addresses for authentication and reset workflows. In some embodiments, a request specifying an uppercase variant of the registered email may be canonicalized to the stored email token, and the system may dispatch the reset notification without requiring manual correction. In some embodiments, documentation and coding engine 807 may record the normalized token, request timestamp, and delivery outcome, and safety module 805 may enforce rate limits and policy checks on reset dispatch.

In some embodiments, the system is configured to enhance search behavior across the content libraries (e.g., forms/modules/bundles library) so that queries match full text entries and return results accordingly. In some embodiments, the system is configured to improve search within a medication training module set (e.g., MedReady) so that keyword queries match both titles and descriptions and require all search terms to match.

In some embodiments, the system is configured to alphabetize dropdowns and lists throughout the assignment workflow to reduce user error. In some embodiments, the system is configured to set the sender name on patient-facing emails to the practice name to improve clarity. In some embodiments, the system is configured to allow clinic users to locate forms that are waiting on other staff members for action. In some embodiments, the system is configured to present patient-facing messaging that explains why a form is locked when prerequisites are unmet. In some embodiments, safety module 805 enforces gating conditions and messaging rules.

In some embodiments, the system is configured to provide in-platform access to staff training videos from the home dashboard (e.g., portal home page) and the staff task list (e.g., Tasks page). In some embodiments, the system is configured to standardize role nomenclature across the user interface so that prior “Nurses” labels appear as “Staff.” In some embodiments, documentation and coding engine 807 is configured to log assignment filters, searches, reminder dispatches, and state changes with timestamps and provenance for auditability.

In some embodiments, interactive patient interface 806 provides an adaptive decision engine summary view 1302. In some embodiments, summary view 1302 may include current parameters, temporal phase indicators from temporal recovery model 1005, and top risk predictions from risk prediction model 1003, as non-limiting examples. In some embodiments, summary view 1302 includes statistical confidence scores to guide emphasis and potential escalation, and includes rationale highlights to assist clinician review and decision-making.

In some embodiments, interactive patient interface 806 includes a module preview/editor 1303. In some embodiments, module preview/editor 1303 enables clinicians to review and modify risk text, animations or simulations, avatar and voice selections, branching logic, tone, and template choices prior to or following delivery. In some embodiments, edits performed in module preview/editor 1303 persist into preference overlay 1006 and propagate through adaptive decision engine 803 to adaptive content engine 804, subject to safety module 805 constraints and approvals.

In some embodiments, the system is configured to provide bundle administration within interactive patient interface 806. In some embodiments, the system is configured to support keyword search for bundles in an assignment workflow page. In some embodiments, the system is configured to restrict bundle creation and editing to authorized administrators and to prevent non admin users from accessing bundle create or edit workflows. In some embodiments, the system is configured to present ordered bundle activities and to allow drag and drop reordering. In some embodiments, the system is configured to display an archive confirmation dialog and to preserve version history when a bundle is archived. In some embodiments, the system is configured to honor bundle sequencing during delivery by adaptive content module 804 and by real time personalization loop 1201. In some embodiments, safety module 805 is configured to enforce governance and role restrictions for bundle management. In some embodiments, documentation and coding engine 807 is configured to log bundle configuration changes, versions, and provenance for auditability. In some embodiments, if a form is unpublished or unmapped due to configuration changes, the system is configured to retain the form within the bundle library and to block assignment until the form is remapped.

In some embodiments, interactive patient interface 806 includes an escalation alerts panel 1304. In some embodiments, escalation alerts panel 1304 is configured to display aggregates of system-generated alerts, including predicted non-adherence, elevated complication risk, abnormal symptom inputs, and rule violations detected by adaptive decision engine 803 or safety module 805. In some embodiments, escalation alerts panel 1304 presents action controls to notify staff, schedule urgent follow-ups, approve triage scripts, or temporarily block content pending review, as non-limiting examples.

In some embodiments, interactive patient interface 806 includes customization settings 1305. In some embodiments, customization settings 1305 are configured to capture physician and institution preferences for tone, language and cultural context, risk emphasis sliders, allowed content types, and escalation thresholds. In some embodiments, customization settings 1305 persist to preference overlay 1006 and are enforced by safety module 805 at runtime.

In some embodiments, the system is configured to enhance assignment navigation within interactive patient interface 806. In some embodiments, the system is configured to allow click through from an assignment record to the corresponding patient record page (e.g., patient record view). In some embodiments, the system is configured to preserve list position and applied filters across tabs and detail views within assignment lists. In some embodiments, the system is configured to present success notifications when reminders are sent and to temporarily disable repeat clicks to prevent duplicate sends. In some embodiments, the system is configured to display status icons for advanced identity verification (e.g., IDV) and gating status (e.g., Blocked By) and to highlight those icons when an assignment becomes active. In some embodiments, documentation and coding engine 807 is configured to log reminder dispatches, navigation events, and state changes with timestamps and provenance. In some embodiments, safety module 805 is configured to enforce consistency and policy rules for navigation and reminder actions.

In some embodiments, interactive patient interface 806 implements approval workflows for clinician edits. In some embodiments, non-limiting approval workflows include version control, change logs, mandatory sign-off checkpoints for sensitive content and documentation, and rollback capabilities to maintain safety and compliance. In some embodiments, safety module 805 applies clinical guardrails to all clinician edits prior to publication, including required phrasing constraints and policy checks.

In some embodiments, the system is configured to provide staff-facing onboarding and training workflows within interactive patient interface 806. In some embodiments, the system is configured to grant staff access to patient-facing eLearning modules for onboarding, reference, and refresher training. In some embodiments, the system is configured to integrate external staff training catalogs via application programming interfaces and data ingestion module 802. In some embodiments, the system is configured to track staff progress and completion of assigned training, to generate status and completion reports with timestamps, and to surface outstanding requirements to supervisors. In some embodiments, documentation and coding engine 807 is configured to log staff training events, maintain audit-ready provenance, and expose administrative views for compliance review. In some embodiments, the system is configured to enforce administrative controls for assignment and access based on role-based permissions. In some embodiments, safety module 805 is configured to govern training policies, approval workflows, and recertification intervals and to block access to certain clinical actions until required training is completed.

In some embodiments, interactive patient interface 806 integrates with electronic medical record (EMR) workflows to streamline documentation and orders. In some embodiments, clinicians may review and sign EMR-ready notes produced by documentation and coding engine 807 and confirm EMR documentation output 808. In some embodiments, interactive patient interface 806 facilitates acknowledgment of alerts and placement of follow-up actions within the clinical workflow.

In some embodiments, the system is configured to manage reminders and notifications within interactive patient interface 806. In some embodiments, the system is configured to send due date reminder emails at defined intervals (e.g., seven and two days before the due date). In some embodiments, the system is configured to create a single combined notification when multiple assignments are issued in one workflow. In some embodiments, the system is configured to allow clinics to set a reminder digest frequency and to adjust that frequency as needed.

In some embodiments, the system is configured to sort assignment lists by due date and to present decline or void notifications to relevant participants in the signing order. In some embodiments, interactive patient interface 806 may implement notification routing policies that align to signing-order indices. In some embodiments, when a signer declines an assignment, the system may dispatch notifications to participants positioned before the declining signer within the signing order. In some embodiments, when a form is voided, notifications may be dispatched to all participants included in the affected signing order. In some embodiments, documentation and coding engine 807 may record signer indices, routing lists, delivery timestamps, and outcomes, and safety module 805 may verify routing policies against institutional rules prior to dispatch.

In some embodiments, the system is configured to require a doctor on record for the episode of care before assignments can be issued. In some embodiments, the system is configured to cease stale patient notifications for irrelevant or older assignments (e.g., assignments older than thirty days). In some embodiments, the system is configured to filter the “assignments due soon” list by date range, assignment name, and assigned patient.

In some embodiments, the system is configured to allow staff to map a carbon copy recipient to a specific role and to select individual users for that mapping. In some embodiments, the system is configured to hide form descriptions from the patient view to reduce confusion. In some embodiments, the system is configured to preserve configuration stability such that changes to titles, role mapping, descriptions, text fields, or signing order do not automatically unpublish forms. In some embodiments, the system is configured to present UI affordances that clarify signer selection and editability and to support adding related patients or care team members to an episode of care.

In some embodiments, documentation and coding engine 807 is configured to record timestamps for reminders and edits and to maintain provenance for audit logging. In some embodiments, safety module 805 is configured to enforce notification policies and signing order rules. In some embodiments, the system is configured to support foreign subtitles for applicable learning modules, including medication training modules (e.g., MedReady).

In some embodiments, interactive patient interface 806 is configured to display decision support visualizations. In some embodiments, non-limiting visualizations include risk trajectories, likelihood-of-event timelines, and guideline or pathway compliance indicators that support evidence-based intervention timing. In some embodiments, interactive patient interface 806 provides operational and quality analytics views that reflect time savings, reduction in repetitive counseling, standardization metrics, and utilization reports for practice and enterprise leadership.

In some embodiments, interactive patient interface 806 provides authentication flows (MFA, biometrics) and continuous presence verification. In some embodiments, identity signals are consumed by data ingestion 802 and decision engine 803 and recorded in documentation logs.

FIG. 14 illustrates various modules of documentation and coding engine 807, in accordance with some embodiments, which may be used for generation of visit-level, billable encounter documentation. In some embodiments, documentation and coding engine 807 receives structured decision signals from adaptive decision engine 803 and interaction records from interactive patient interface 806, and produces electronic medical record (EMR) documentation output 808. In some embodiments, documentation and coding engine 807 is configured to assemble audit-ready records that reflect patient education delivered, interaction outcomes, and clinical reasoning elements suitable for insertion into an EMR.

In some embodiments, an interaction log module 1401 captures complete, timestamped records of patient interactions during module delivery. In some embodiments, interaction log module 1401 records quiz results, branching choices, symptom inputs, and engagement or comprehension metrics, as non-limiting examples, and associates each record with applicable decision rationales and safety module 805 actions.

In some embodiments, an education summary generator module 1402 compiles a structured summary of personalized education delivered. In some embodiments, education summary generator module 1402 enumerates sections and topics presented, risks and alternatives discussed, comprehension checkpoints achieved or missed, and language or literacy adaptations applied, as non-limiting examples.

In some embodiments, a medical decision-making (MDM) extractor module 1403 identifies and documents clinical decision elements. In some embodiments, MDM extractor module 1403 records problems addressed, data reviewed, and risk of complications or morbidity aligned to applicable coding frameworks.

In some embodiments, a time-based coding calculator module 1404 aggregates time on task for educational interactions and documentation review. In some embodiments, time-based coding calculator module 1404 relies on interaction log 1401 timestamps to support compliant billing documentation.

In some embodiments, a consent support extractor module 1405 assembles consent-support narratives. In some embodiments, consent support extractor module 1405 compiles required risk disclosures, alternative options, expected outcomes, and confirmation prompts that demonstrate understanding and capture digital acknowledgments where available.

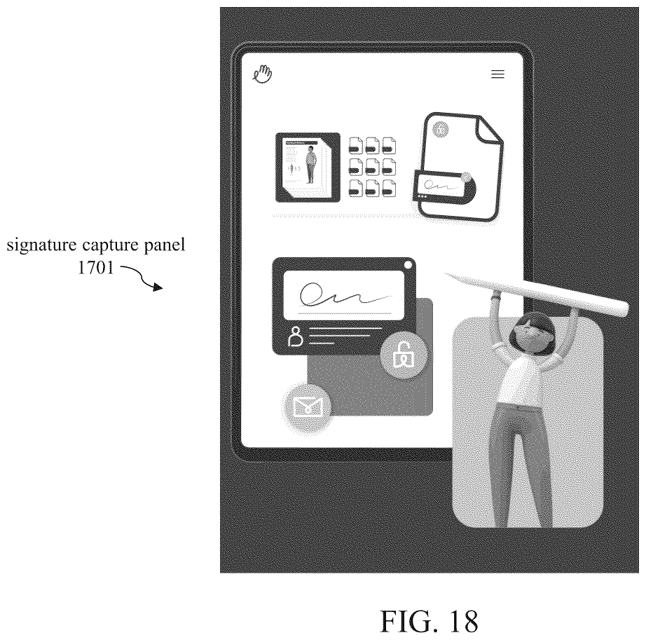

In some embodiments, consent support extractor module 1405 operates within documentation and coding engine 807. In some embodiments, the system is configured to capture digital consent artifacts within interactive patient interface 806, including acknowledgments, initials, and electronic signatures. As illustrated in FIG. 18, in some embodiments, interactive patient interface 806 is configured to render a signature capture panel 1801 comprising a signer card, a signature input region, an identity badge that reflects the authenticated signer, and a privacy lock indicator that reflects encryption and role-based access status. In some embodiments, documentation and coding engine 807 is configured to compile signature artifacts together with signer identity, authentication method, time-of-sign event, and consent context fields, and to associate each compiled artifact with the envelope identifier and cataloged template key. In some embodiments, safety module 805 is configured to enforce required phrasing and mandatory fields prior to acceptance of the signature artifact and to block submission until gating conditions are satisfied. In some embodiments, safety module 805 is configured to enforce required phrasing, mandatory fields, and institutional policy checks prior to submission.

In some embodiments, documentation and coding engine 807 is configured to compile consent artifacts together with comprehension evidence, timestamps, signer identity, and authentication method and to produce EMR documentation output 808 with provenance. In some embodiments, the system is configured to standardize and track participant progress, comprehension, and consent for clinical trials and to align those workflows with provider views in interactive patient interface 806.

In some embodiments, an EMR note compiler module 1406 formats documentation for insertion into an EMR. In some embodiments, EMR note compiler module 1406 produces outputs compatible with clinical document architecture (CDA) and fast healthcare interoperability resources (FHIR) DocumentReference, as non-limiting examples, and applies payer-specific pre-authorization fields where applicable.

In some embodiments, the system is configured to present combined envelopes of assigned forms within interactive patient interface 806 and to enforce signing orders. In some embodiments, interactive patient interface 806 may gate the presentation of a signing control element by the signing order. In some embodiments, the signing control element (“Sign” button) may be displayed only when the current user occupies the active signer index for the envelope. In some embodiments, safety module 805 may validate the active signer index prior to rendering, and documentation and coding engine 807 may log visibility state changes, signer indices, and associated role permissions for auditability. In some embodiments, the system is configured to limit assignment of signature steps to active staff users according to role-based permissions.

In some embodiments, the system is configured to support markup workflows and to dispatch responsive notifications to affected signers when markup is applied. In some embodiments, application of a markup action to an assigned form may trigger deterministic state transitions. In some embodiments, progress status may be reset to “Waiting” and assignment status may be reset to “Assigned” upon markup completion, and documentation and coding engine 807 may update the assignment timestamp field with the completion time of the markup event. In some embodiments, provider-facing views within interactive patient interface 806 may reflect the updated statuses and timestamps, and safety module 805 may enforce consistency checks prior to re-publication of revised artifacts.

In some embodiments, the system is configured to send a completed form email that links directly to a PDF of finalized forms and to append a certificate of completion generated by documentation and coding engine 807. In some embodiments, the system is configured to prepopulate fields for signers later in the signing order when demographic information is available and predictable. In some embodiments, the system is configured to allow staff to unlock a gated form at their discretion when a module prerequisite is incomplete. In some embodiments, the system is configured to select and default advanced authentication options at the practice or template level. In some embodiments, the system is configured to add carbon copy recipients who receive completed forms without being required to sign.

In some embodiments, the system is configured to allow downloading of in-progress forms for review. In some embodiments, the system is configured to ingest existing form templates, to render legally enforceable electronic signatures, and to automatically deliver completed forms to a patient's EMR record. In some embodiments, data ingestion module 802 may be configured to ingest patient-facing form templates in multiple languages and to normalize the ingested artifacts for downstream eSign workflows. In some embodiments, optical character recognition and natural language processing pipelines may extract field structures and semantic mappings from non-English templates, and normalization module 903 may align extracted fields to internal schemas with language tags, provenance, and confidence metadata.

In some embodiments, the system is configured to present iconography that reflects eSign choices and states. In some embodiments, documentation and coding engine 807 is configured to record signing events, authentication details, timestamps, and provenance and to include those artifacts in EMR documentation output 808. In some embodiments, safety module 805 may enforce template governance rules and required phrasing checks in the source language prior to assignment, and documentation and coding engine 807 may persist language identifiers with completed envelopes to support audit and EMR insertion.

In some embodiments, documentation and coding engine 807 applies quality and compliance checks across modules 1401-1406. In some embodiments, quality and compliance checks verify completeness, institutional policy alignment, required phrasing, and clinical or legal guardrails, and may request remediation before producing EMR documentation output 808. In some embodiments, the system is configured to enforce completion safeguards so that all required forms are completed and signed before progression.

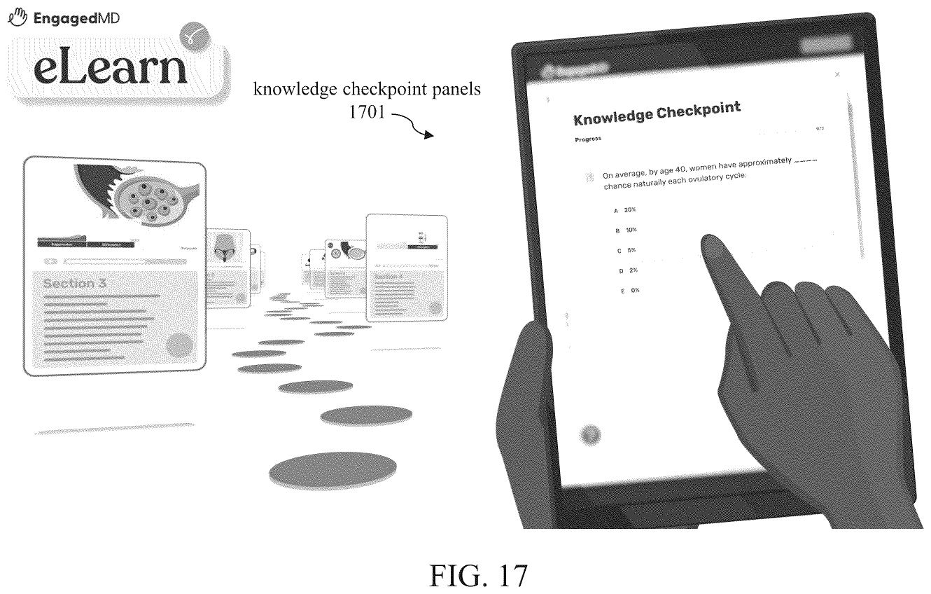

In some embodiments, the system is configured to embed knowledge checks within education modules and to verify comprehension prior to consent or scheduling steps. As illustrated in FIG. 17, in some embodiments, interactive patient interface 806 is configured to present knowledge-checkpoint panels that include a question prompt, a multiple-choice response set, and a session-level progress indicator. In some embodiments, adaptive content engine 804 is configured to record per-attempt inputs, correctness outcomes, time on task, and progress-meter values as structured fields, and to apply gating thresholds that require minimum comprehension prior to advancing to subsequent segments or initiating consent steps. In some embodiments, safety module 805 is configured to insert mandatory checkpoints when policy or risk thresholds are met, and documentation and coding engine 807 is configured to persist checkpoint results, gating decisions, and associated timestamps with module identifiers for inclusion in EMR documentation output 808.

In some embodiments, the system is configured to maintain a comprehensive audit trail, including event timestamps, actor identity, and action outcomes. In some embodiments, the system is configured for cloud-based integration with clinical tools and to support personalized workflows aligned to practice preferences. In some embodiments, the system is configured to apply role-based access controls, encryption in transit and at rest, audit logging, and field level provenance in alignment with safety module 805 governance and documentation and coding engine 807 practices. In some embodiments, these features are configured to reduce human error and to improve compliance.

In some embodiments, safety module 805 constrains documentation phrasing prior to EMR insertion. In some embodiments, safety module 805 enforces physician-approved templates, institutional guidelines, and required language for risk or consent-related content and logs allow, modify, block, or escalate decisions for auditability.

In some embodiments, safety module 805 is configured to enforce role-gated visibility for identity verification (IDV) and short message service (SMS) authorization failures. In some embodiments, determination module 206 may evaluate a signer's role and signing-order position and may suppress display of failure views to non-privileged users while enabling access for users holding required permissions. In some embodiments, interactive patient interface 806 may render failure views only when a user's role satisfies access controls enforced by safety module 805, and documentation and coding engine 807 may record the gate decision, actor role, failure classification, and timestamp in association with the affected envelope identifier. In some embodiments, documentation and coding engine 807 incorporates security and privacy controls aligned with protected health information (PHI) requirements. In some embodiments, security and privacy controls include encryption in transit and at rest, role-based access controls, PHI audit logging, and field-level provenance for source attribution.

In some embodiments, documentation and coding engine 807 writes continuously during real-time personalization loop 1201 and finalizes notes upon module completion. In some embodiments, documentation and coding engine 807 minimizes clinician burden by preparing EMR-ready narratives that can be reviewed and signed within clinical workflows.

In some embodiments, documentation and coding engine 807 employs multi-agent artificial intelligence (AI) components to assist with summarization, code extraction, and consent narrative drafting. In some embodiments, AI-assisted outputs are validated by rule-based checks prior to compilation and subject to approval workflows and versioning controls.

In some embodiments, documentation and coding engine 807 exposes population-level documentation analytics for quality improvement and operational reporting. In some embodiments, documentation analytics include documentation completeness rates, consent-support coverage, time savings, and coding accuracy metrics, and are aggregated to preserve patient privacy.

FIG. 15 illustrates multi-specialty deployment configurations of compliance engine 200, in accordance with some embodiments. In some embodiments, compliance engine 200 serves as a central hub that supports specialty modules for urology 1501, cardiology 1502, gastroenterology 1503, oncology 1504, and obstetrics/gynecology (OB/GYN) 1505, as non-limiting examples. In some embodiments, each specialty module uses the same pipeline provided by data ingestion module 802, adaptive decision engine 803, adaptive content engine 804, safety module 805, interactive patient interface 806, and documentation and coding engine 807, as described above.

In some embodiments, urology module 1501 provides educational pathways for procedures and conditions such as vasectomy, benign prostatic hyperplasia (BPH), prostate-specific antigen (PSA) counseling, hematuria evaluation, and kidney stone management, as non-limiting examples. In some embodiments, urology module 1501 includes specialty content templates and animations generated by multimedia module 1102, risk parameters modeled within adaptive decision engine 803, branching trees configured within branching decision engine 1103, and consent-support elements assembled by documentation and coding engine 807.

In some embodiments, cardiology module 1502 provides educational pathways for heart failure management, percutaneous coronary intervention (PCI) preparation, atrial fibrillation (AFib) counseling, and cardiac rehabilitation, as non-limiting examples. In some embodiments, cardiology module 1502 incorporates anticoagulation-specific perioperative instructions and medication-related risk modeling within adaptive decision engine 803, and supports integration of wearable and internet-of-things (IoT) signals such as heart rate and blood pressure to inform personalization and alerts.

In some embodiments, gastroenterology module 1503 provides educational pathways for colonoscopy preparation, reflux and gastroesophageal reflux disease (GERD) management, and inflammatory bowel disease (IBD) counseling, as non-limiting examples. In some embodiments, gastroenterology module 1503 uses scheduling-aware adjustments to intensify preparation content as procedure dates approach and applies multilingual and literacy adaptations via lingual adaptation module 1104 to improve clarity for stepwise preparation.

In some embodiments, the system is configured to assemble and present colonoscopy and upper endoscopy modules that include introduction, bowel preparation, how the procedure works, anesthesia options, risks, and recovery guidance. In some embodiments, the system is configured to present bowel preparation kit instructions and to emphasize preparation quality using comprehension checkpoints and reminders. In some embodiments, the system is configured to intensify preparation content based on scheduling updates received by data ingestion module 802 and real time personalization loop 1201. In some embodiments, the system is configured to apply multilingual and literacy adaptations via lingual adaptation module 1104. In some embodiments, the system is configured to allow staff to assign relevant gastroenterology content through interactive patient interface 806 and to record delivery and comprehension outcomes in documentation and coding engine 807. In some embodiments, previously shown high level gastroenterology application concepts are consolidated here as written system features.

In some embodiments, oncology module 1504 provides educational pathways for biopsy processes, cancer staging explanations, chemotherapy preparation, and survivorship planning, as non-limiting examples. In some embodiments, oncology module 1504 responds to pathology updates received through data ingestion module 802 and adaptive decision engine update module 905, and invokes symptom triage and escalation scripts through interactive patient interface 806 when concerning inputs are detected by adaptive decision engine 803 or safety module 805.

In some embodiments, the system is configured to present oncology onboarding and demystification modules that explain diagnosis details and care pathways. In some embodiments, the system is configured to introduce the care team and to assemble surgical, radiation, and medical treatment segments with appropriate durations. In some embodiments, the system is configured to insert comprehension checkpoints and to capture consent support elements throughout the modules. In some embodiments, adaptive content module 804 is configured to assemble the oncology content; interactive patient interface 806 is configured to deliver the content; safety module 805 is configured to enforce required disclosures and phrasing; and documentation and coding engine 807 is configured to record outcomes and produce EMR ready documentation.

In some embodiments, OB/GYN module 1505 provides educational pathways for hysterectomy, pregnancy care, and contraception counseling, as non-limiting examples. In some embodiments, OB/GYN module 1505 applies surgeon-specific technique preferences via preference overlay 1006 and generates day-specific recovery trajectories using temporal recovery model 1005 to tailor postoperative guidance.

In some embodiments, the system is configured to assemble and present orthopedics specialty education, including total knee replacement, rotator cuff repair, total hip replacement, and non-surgical treatments. In some embodiments, the system is configured to verify compliance with assigned education and to standardize digital education across providers and locations. In some embodiments, the system is configured to reduce patient inquiries by presenting trustworthy, structured content prior to clinic visits. In some embodiments, adaptive content module 804 is configured to assemble the orthopedics modules. In some embodiments, interactive patient interface 806 is configured to deliver the modules across endpoints. In some embodiments, safety module 805 is configured to enforce clinical guardrails and required phrasing. In some embodiments, documentation and coding engine 807 is configured to record education and consent support outcomes and to produce audit-ready, EMR-ready documentation.

In some embodiments, each specialty module is configured to include tailored risk models within adaptive decision engine 803, specialty-specific pathway logic and branching within adaptive content engine 804, content libraries and animations within multimedia module 1102, language and cultural adaptations within lingual adaptation module 1104, and documentation templates within documentation and coding engine 807. In some embodiments, safety module 805 incorporates applicable specialty society guidelines and institutional policies and enforces clinical guardrails and required phrasing prior to delivery and documentation.

In some embodiments, physician and institution customization is applied across specialties using preference overlay 1006. In some embodiments, preference overlay 1006 persists provider tone, risk framing, surgical approach, equipment or technique selections, and language preferences, which are enforced by safety module 805 and reflected in adaptive content engine 804 outputs. In some embodiments, scheduling-aware flows intensify preparation content and reminders as procedure dates approach and personalize post-procedure follow-ups with day-specific instructions and red-flag symptom education, as non-limiting examples.

In some embodiments, specialty modules support augmentation by wearable and IoT inputs, such as ambulation metrics for orthopedic-style recovery, heart rate or blood pressure for cardiology, and temperature for oncology neutropenia risk, as non-limiting examples. In some embodiments, these inputs feed data ingestion module 802 and adaptive decision engine 803, and adjust loop behavior within real-time personalization loop 1201. In some embodiments, offline and low-bandwidth operation is supported across specialties by caching modules and deferring synchronization with conflict resolution on reconnect. In some embodiments, specialty modules implement autonomous triage and escalation rules vetted by safety module 805 and surfaced in provider-facing views within interactive patient interface 806. In some embodiments, adaptive decision engine 803 proposes follow-ups or orders, or temporarily blocks unsafe pathways pending clinician review, and logs all actions for auditability and documentation.

In some embodiments, specialty-level analytics are exposed through interactive patient interface 806 and documentation and coding engine 807. In some embodiments, analytics include outcome trends, preparation compliance rates, documentation completeness metrics, and alert volumes, and are aggregated to support quality improvement and operational reporting while preserving patient privacy. In some embodiments, deployment of specialty modules at enterprise scale standardizes education across providers and locations, reduces call volume, improves preparation compliance, improves coding accuracy, and supports measurable return on investment within clinical operations.

In some embodiments, compliance engine 200 is configured to execute computational analysis using one or more predictive models and structured artificial intelligence (AI) architectures as described above. In some embodiments, these architectures include, without limitation, convolutional neural networks (CNNs), recurrent neural networks (RNNs), long short-term memory (LSTM) networks, transformer-based language models, autoencoders, support vector machines (SVMs), decision trees, and ensemble methods such as random forests and gradient boosting. In some embodiments, probabilistic models such as Bayesian networks and confidence-calibration methods (e.g., Platt scaling or isotonic regression) are employed to improve reliability of predictions. In some embodiments, multiple predictive models and/or AI models are deployed in combination or selected as alternatives based on the clinical application to achieve optimal performance and safety.

In some embodiments, the system employs transformer-based natural-language models to interpret unstructured electronic medical record (EMR) documents and clinical policies. In some embodiments, a transformer model is fine-tuned on domain-specific corpora that include clinical notes, operative reports, referral letters, and institutional guideline text. In some embodiments, training uses attention mechanisms and tokenization suitable for medical language, and the trained model, comprising weights, tokenizer, and configuration, is serialized and stored on non-transitory computer-readable media. In some embodiments, during inference one or more processors tokenize input text, produce embeddings or logits, and decode entities, relations, or summaries that are consumed by adaptive decision engine 803 for condition-to-module mapping, risk phrasing, and consent-support assembly.

In some embodiments, the system employs recurrent neural networks and/or long short-term memory (LSTM) networks to process clinical time-series data and temporal event sequences. In some embodiments, inputs include laboratory trends, vital-sign streams, medication administration timelines, appointment schedules, and recovery day counts. In some embodiments, training uses backpropagation through time on historical sequences framed for forecasting tasks (e.g., next-day lab trajectory, time-to-event forecasts), and the trained network is serialized for deployment. In some embodiments, at inference, recent time windows are fed through recurrent cells to generate short-term forecasts and phase indicators that update temporal recovery model 1005 and scheduling-aware urgency logic in adaptive decision engine 803.

In some embodiments, the system employs ensemble risk-prediction models to estimate clinically relevant outcomes. In some embodiments, gradient boosting, random forests, and regularized logistic regression are trained on feature sets that include demographics, diagnoses, comorbidities, labs, imaging flags, medications, and interaction-derived engagement signals. In some embodiments, outputs include probabilities for complications, emergency department (ED) visits, hospital readmissions, non-adherence to instructions, appointment cancellations, pain trajectories, and recovery timelines, as non-limiting examples. In some embodiments, prediction probabilities are calibrated via Platt scaling or isotonic regression to produce reliable thresholds for emphasis and escalation in adaptive decision engine 803.

In some embodiments, the system employs Bayesian inference or Bayesian networks to capture probabilistic dependencies among clinical variables. In some embodiments, nodes represent comorbidities, medications, planned procedures, phase of care, and observed signals from EMR and sensors, and edges represent conditional relationships learned from historical data or defined by expert rules. In some embodiments, probabilistic inference computes posterior probabilities given observed evidence (e.g., new lab abnormality) and informs decision outputs such as branch selection, disclosure emphasis, and alert prioritization.

In some embodiments, the system employs autoencoders to perform dimensionality reduction and anomaly detection on multivariate EMR streams. In some embodiments, an encoder-decoder architecture is trained on “normal” distributions of labs, vitals, and scheduling patterns; reconstruction error is computed at inference to flag deviations that may indicate emergent risk, documentation inconsistencies, or data quality issues. In some embodiments, latent representations produced by the encoder are used as compact features for downstream risk-prediction ensembles and temporal forecasting models.

In some embodiments, the system employs decision trees to classify decision states, route branching logic, and select triage actions. In some embodiments, trees are trained on labeled outcomes (e.g., adherence events, escalation cases) with splits selected via information gain or Gini impurity; pruning reduces overfitting. In some embodiments, decision trees are serialized into nested structures and evaluated at runtime to determine module sequencing, checkpoint insertion, and escalation triggers. In some embodiments, gradient boosting extends these trees to improve robustness for classification and regression tasks used by adaptive decision engine 803.

In some embodiments, the system employs reinforcement-learning policies to optimize real-time sequencing and interaction cadence within the personalization loop. In some embodiments, the policy's state includes current decision parameters, content history, engagement metrics, and proximity to scheduled events; rewards are defined to maximize comprehension, adherence, and risk-reduction while minimizing time burden. In some embodiments, training occurs offline with logged trajectories and is updated under controlled, auditable workflows; at runtime, the learned policy proposes module adjustments that are reviewed under safety module 805 guardrails before delivery.

In some embodiments, the system employs recommendation and learning-to-rank models to select and order educational modules. In some embodiments, features include decision-state vectors from adaptive decision engine 803, historical completion and comprehension signals, and physician or institution preference overlay 1006 parameters. In some embodiments, the ranking model outputs an ordered list of modules and disclosures that the content assembly controller 1101 composes and the branching decision engine 1103 adapts during delivery.

In some embodiments, the system employs confidence-calibration and meta-monitoring models to supervise predictive reliability and detect drift. In some embodiments, calibration models adjust raw probabilities to align with observed outcome frequencies, and meta-monitoring tracks performance metrics (e.g., Brier score, area under the curve) to trigger retraining or rollback when data distributions shift. In some embodiments, calibration thresholds are logged and enforced by safety module 805 to gate escalations and mandatory checkpoint insertions.

In some embodiments, the system employs multi-agent orchestration models to coordinate specialized AI agents for content generation, clinical validation, prediction, and documentation assembly. In some embodiments, an agent-coordination module manages agent roles, merges outputs, and routes artifacts through safety module 805 allow/modify/block/escalate checks before presentation in interactive patient interface 806 or insertion into EMR documentation output 808. In some embodiments, agent lifecycles are versioned, auditable, and subject to approval workflows consistent with institutional policy.

In some embodiments, the system employs natural language processing (NLP) and named entity recognition (NER) models to extract clinical entities, relations, and concepts from unstructured electronic medical record (EMR) text such as clinical notes, operative reports, referral letters, and institutional policies. In some embodiments, these models are fine-tuned on domain-specific corpora and produce structured fields (e.g., diagnoses, medications, labs, procedures, dates) that are consumed by data ingestion module 802 and adaptive decision engine 803 for normalization and condition-to-module mapping.

In some embodiments, the system employs optical character recognition (OCR) models to convert scanned documents and portable document format (PDF) attachments into machine-readable text. In some embodiments, OCR outputs are passed through NLP and NER pipelines to recover clinical entities and provenance, enabling ingestion and downstream decisioning from otherwise non-searchable files.

In some embodiments, the system employs ontology mapping and disambiguation models to align free-text terms and local codes to standardized vocabularies including Systematized Nomenclature of Medicine (SNOMED), Logical Observation Identifiers Names and Codes (LOINC), International Classification of Diseases (ICD), and current procedural terminology (CPT). In some embodiments, the mapping models use semantic similarity, context windows, and code co-occurrence statistics to produce normalized identifiers with confidence scores and source lineage for auditability.

In some embodiments, the system employs computer-vision models for gaze estimation, pose detection, and presence verification to assess attention and engagement during module delivery. In some embodiments, these models operate on camera streams at interactive patient interface 806 to detect whether the patient is present, facing the screen, and maintaining visual focus on key content regions, and produce engagement metrics that inform checkpoint cadence and module adjustments.

In some embodiments, the system employs voice-activity detection (VAD) and ambient-noise classification models to monitor audio context during delivery and interaction. In some embodiments, VAD identifies speech segments and silence, and ambient noise classification detects disruptive environments (e.g., loud background) so that the system can pause, repeat, or switch modalities to preserve comprehension.

In some embodiments, the system employs machine translation models to render content into multiple languages with clinically appropriate terminology. In some embodiments, translation outputs are post-processed by readability and text-simplification models to target grade-level and clarity, ensuring language and literacy adaptation for diverse patient populations.

In some embodiments, the system employs text-to-speech (TTS) models to generate multilingual voice output with correct pronunciation of clinical terms. In some embodiments, lip-sync and phoneme-alignment models synchronize avatar mouth movements to TTS audio, producing coherent audiovisual explanations that match selected personas and improve engagement.

In some embodiments, the system employs style and tone-transfer models to adapt phrasing and delivery to physician or institution preferences captured in preference overlay 1006. In some embodiments, these models adjust formal vs. conversational style, emphasis, and cultural references while preserving factual accuracy enforced by safety module 805.

In some embodiments, the system employs generated-text quality control models to detect factuality issues, hallucinations, and toxicity in automated explanations and consent-support text. In some embodiments, these models score outputs and route low-confidence or non-conforming text through safety module 805 for modification or blocking prior to delivery or documentation.

In some embodiments, the system employs contraindication-interaction models to identify unsafe instruction combinations based on medications, conditions, and planned procedures. In some embodiments, these models use learned interaction graphs augmented by rules to flag risks (e.g., anticoagulant use before biopsy), insert required disclosures, and trigger escalation when thresholds are met.

In some embodiments, the system employs signal-processing and anomaly-detection models for wearable and internet-of-things (IoT) streams, including heart rate, blood pressure, temperature, and ambulation. In some embodiments, these models smooth noise, detect clinically relevant deviations, and produce alerts and personalization cues for adaptive decision engine 803.

In some embodiments, the system employs personalized threshold-adaptation models that adjust alert and guidance thresholds to patient baselines and risk profiles. In some embodiments, thresholds evolve with observed signals and decision-state changes to balance sensitivity and specificity for escalation and messaging.

In some embodiments, the system employs summarization natural-language generation (NLG) models to compile education delivered, interaction outcomes, and comprehension evidence into documentation drafts. In some embodiments, the NLG outputs are validated by rule-based checks and safety module 805 before insertion by documentation and coding engine 807.

In some embodiments, the system employs code extraction and suggestion models to identify appropriate ICD, CPT, and modifier codes from decision logs, content delivery metadata, and medical decision-making (MDM) elements. In some embodiments, these models assist coding accuracy and consistency and are subject to policy verification prior to EMR documentation output 808.

In some embodiments, the system employs consent-narrative assembly models to draft risk disclosures, alternatives, expected outcomes, and confirmation prompts. In some embodiments, consent narratives are constrained by institution-specific rules and required phrasing enforced by safety module 805, and are linked to comprehension evidence recorded at interactive patient interface 806.

In some embodiments, the system employs drift-detection and retraining-scheduler models to monitor predictive performance, calibration, and data distributions over time. In some embodiments, performance triggers initiate offline retraining or fine-tuning under auditable workflows with approval steps, versioning, and rollback controls to maintain safety.

In some embodiments, the system employs explainability models or methods, such as Shapley additive explanations (SHAP) or local interpretable model-agnostic explanations (LIME), to generate feature-importance and rationale views for clinician review. In some embodiments, explanation artifacts are surfaced in adaptive decision engine summary view 1302 to support transparency and trust.

In some embodiments, the system employs survival-analysis models to estimate time-to-event outcomes for recovery trajectories and complication risks. In some embodiments, these models complement recurrent forecasting by providing hazard estimates that guide day-specific instructions and escalation thresholds.

In some embodiments, the system employs federated-learning and edge-learning variants for privacy and low-latency contexts. In some embodiments, model updates are trained locally on device or within institutional boundaries and aggregated centrally under governance, with inference executed at the edge where appropriate.

In some embodiments, the system employs scheduling-optimization models to plan preparation steps, reminders, and follow-up cadence. In some embodiments, optimization policies consider proximity to procedures, predicted adherence, and clinic load, and produce schedules that are adapted in real time by the personalization loop and validated by safety module 805.

As used herein, the terms “computer engine” and “engine” identify at least one software component and/or a combination of at least one software component and at least one hardware component which are designed/programmed/configured to manage/control other software and/or hardware components (such as the libraries, software development kits (SDKs), objects, and the like).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Computer-related systems, computer systems, and systems, as used herein, include any combination of hardware and software. Examples of software may include software components, programs, applications, operating system software, middleware, firmware, software modules, routines, subroutines, functions, methods, procedures, software interfaces, application program interfaces (API), instruction sets, computer code, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

For the purposes of this disclosure a module is a software, hardware, or firmware (or combinations thereof) system, process or functionality, or component thereof, that performs or facilitates the processes, features, and/or functions described herein (with or without human interaction or augmentation). A module can include sub-modules. Software components of a module may be stored on a computer readable medium for execution by a processor. Modules may be integral to one or more servers or be loaded and executed by one or more servers. One or more modules may be grouped into an engine or an application.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores,” may be stored on a tangible, machine readable medium and supplied to various customers or manufacturing facilities to load into the fabrication machines that make the logic or processor. Of note, various embodiments described herein may, of course, be implemented using any appropriate hardware and/or computing software languages (e.g., C++, Objective-C, Swift, Java, JavaScript, Python, Perl, QT, and the like).

For example, exemplary software specifically programmed in accordance with one or more principles of the present disclosure may be downloadable from a network, for example, a website, as a stand-alone product or as an add-in package for installation in an existing software application. For example, exemplary software specifically programmed in accordance with one or more principles of the present disclosure may also be available as a client-server software application, or as a web-enabled software application. For example, exemplary software specifically programmed in accordance with one or more principles of the present disclosure may also be embodied as a software package installed on a hardware device.

For the purposes of this disclosure the term “user,” “subscriber” “consumer” or “customer” should be understood to refer to a user of an application or applications as described herein and/or a consumer of data supplied by a data provider. By way of example, and not limitation, the term “user” or “subscriber” can refer to a person who receives data provided by the data or service provider over the Internet in a browser session, or can refer to an automated software application which receives the data and stores or processes the data. Those skilled in the art will recognize that the methods and systems of the present disclosure may be implemented in many manners and as such are not to be limited by the foregoing exemplary embodiments and examples. In other words, functional elements being performed by single or multiple components, in various combinations of hardware and software or firmware, and individual functions, may be distributed among software applications at either the client level or server level or both. In this regard, any number of the features of the different embodiments described herein may be combined into single or multiple embodiments, and alternate embodiments having fewer than, or more than, all of the features described herein are possible.

Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, as well as those variations and modifications that may be made to the hardware or software or firmware components described herein as would be understood by those skilled in the art now and hereafter.

Furthermore, the embodiments of methods presented and described as flowcharts in this disclosure are provided by way of example in order to provide a more complete understanding of the technology. The disclosed methods are not limited to the operations and logical flow presented herein. In some embodiments, the order of the various operations is altered and in which sub-operations described as being part of a larger operation are performed independently.

The previous detailed description is to be read with reference to the figures, in which like elements in different figures have like reference numerals. The figures, which are not necessarily to scale, depict some embodiments and are not intended to limit the scope of embodiments of the system.

Any of the operations described herein that form part of the system are useful machine operations. The system also relates to a device or an apparatus for performing these operations. All flowcharts presented herein represent computer implemented steps and/or are visual representations of algorithms implemented by the system. The apparatus can be specially constructed for the required purpose, such as a special purpose computer. When defined as a special purpose computer, the computer can also perform other processing, program execution or routines that are not part of the special purpose, while still being capable of operating for the special purpose. Alternatively, the operations can be processed by a general-purpose computer selectively activated or configured by one or more computer programs stored in the computer memory, cache, or obtained over a network. When data is obtained over a network the data can be processed by other computers on the network, e.g., a cloud of computing resources.

The embodiments of the system can also be defined as a machine that transforms data from one state to another state. The data can represent an article, that can be represented as an electronic signal and electronically manipulate data. The transformed data can, in some cases, be visually depicted on a display, representing the physical object that results from the transformation of data. The transformed data can be saved to storage generally, or in particular formats that enable the construction or depiction of a physical and tangible object. In some embodiments, the manipulation can be performed by a processor. In such an example, the processor thus transforms the data from one thing to another. Still further, some embodiments include methods that can be processed by one or more machines or processors that can be connected over a network. Each machine can transform data from one state or thing to another, and can also process data, save data to storage, transmit data over a network, display the result, or communicate the result to another machine. Computer-readable storage media, as used herein, refers to physical or tangible storage (as opposed to signals) and includes without limitation volatile and non-volatile, removable and non-removable storage media implemented in any method or technology for the tangible storage of information such as computer-readable instructions, data structures, program modules or other data.

Although method operations are presented in a specific order according to some embodiments, the execution of those steps do not necessarily occur in the order listed unless explicitly specified. Also, other housekeeping operations can be performed in between operations, operations can be adjusted so that they occur at slightly different times, and/or operations can be distributed in a system which allows the occurrence of the processing operations at various intervals associated with the processing, as long as the processing of the overlay operations are performed in the desired way and result in the desired system output.

It will be appreciated by those skilled in the art that while the system has been described above in connection with particular embodiments and examples, the system is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein. Various features and advantages of the system are set forth in the following claims.

We claim:

1. A system comprising:
one or more computers comprising one or more processors and one or more non-transitory computer readable media, the one or more non-transitory computer readable media storing program instructions that when executed cause the one or more processors to:
execute, via the one or more processors, an identification of a user using at least one sensor;
associate, via the one or more processors, a medical record with the user based on the identification;
determine, via the one or more processors, one or more content modules to be displayed on a client device based on the medical record;
determine, via the one or more processors, compliance requirements for each of the one or more content modules;
execute monitoring protocols while each content module is being displayed on a client device;
determine if the compliance requirements are being met based on the monitoring protocols; and
control the client device when noncompliance is detected.

2. The system of claim 1,
wherein identifying the user includes continuous monitoring while the one or more content modules are being displayed.

3. The system of claim 2,
wherein the continuous monitoring includes executing a real-time presence tracking using one or more sensors.

4. The system of claim 3,
wherein artificial intelligence is used to execute the real-time presence tracking.

5. The system of claim 1,
wherein identifying the compliance requirements includes identifying a procedure type.

6. The system of claim 5,
wherein identifying the compliance requirements includes identifying a jurisdiction of the procedure type.

7. The system of claim 6,
where identifying the compliance requirements includes executing rule-based systems in conjunction with one or more artificial intelligence models to determine regulations for specific compliance requirements associated with the jurisdiction.

8. The system of claim 1,
wherein the monitoring protocols include calculating gaze coordinates of a user gaze to determine whether the user is looking at a relevant screen area.

9. The system of claim 8,
wherein the monitoring protocols include assessing attention span by analyzing a duration of the user gaze toward the client device.

10. The system of claim 9,
wherein the monitoring protocols include calculating an engagement score based on sensor data.

11. The system of claim 1,
wherein determining whether the compliance requirements are being met includes comparing an analysis result for a monitored parameter to a compliance threshold value.

12. The system of claim 11,
wherein determining whether the compliance requirements are being met includes implementing only the monitoring protocols associated with the compliance requirements.

13. The system of claim 12,
wherein determining whether the compliance requirements are being met includes flagging low engagement when multiple signals suggest inattention.

14. The system of claim 13,
wherein controlling the client device includes automatically pausing content when a user's gaze moves away from a screen.

15. The system of claim 14,
wherein controlling the client device includes presenting interactive activities responsive to a drop in engagement.

16. The system of claim 15,
wherein controlling the client device includes personalized content delivery responsive to a noncompliance determination.

17. The system of claim 16,
wherein controlling the client device includes a module recalibration that adjusts one or more of content selection, sequencing, segment length, and tone.

18. The system of claim 1,
wherein updating portions of a medical database includes compiling monitoring results and inserting the monitoring results into the medical record.

19. A system comprising:
one or more computers comprising one or more processors and one or more non-transitory computer readable media, the one or more non-transitory computer readable media storing program instructions that when executed cause the one or more processors to:
execute, via the one or more processors, an identification of a user using at least one sensor;
associate, via the one or more processors, a medical record with the user based on the identification;
generate, via the one or more processors, structured clinical questioning based on the medical record;
present, via the one or more processors, the structured clinical questioning through an interactive patient interface on a client device;
capture, via the one or more processors, patient responses to the structured clinical questioning;
perform, via the one or more processors, symptom intake and clinical assessment based on the patient responses;
route, via the one or more processors, clinical assessment results to provider-facing views for clinician review; and
execute, via the one or more processors, escalation workflows when predetermined clinical thresholds are exceeded.

20. A system comprising:
one or more computers comprising one or more processors and one or more non-transitory computer readable media, the one or more non-transitory computer readable media storing program instructions that when executed cause the one or more processors to:
execute, via the one or more processors, an identification of a user using at least one sensor;
associate, via the one or more processors, a medical record with the user based on the identification;
capture, via the one or more processors, timestamped interaction data during clinical encounters;
extract, via the one or more processors, medical decision-making elements from the interaction data using a medical decision-making extractor module;
calculate, via the one or more processors, time-based coding parameters using a time-based coding calculator module;
compile, via the one or more processors, encounter documentation using a documentation and coding engine; generate, via the one or more processors, audit-ready records with comprehensive provenance tracking; and
write, via the one or more processors, the encounter documentation to an electronic medical record system.
